(19) United States
(12) Patent Application Publication
(10) Pub. No.: US 2007/0293544 A1
(43) Pub. Date: Dec. 20, 2007

(54) Novel 4-Arylamino Pyridone Derivatives as Mek Inhibitors for the Treatment of Hyper-Proliferative Disorders

(76) Inventors: Ulrich Abel, Heidelberg (DE); Holger Deppe, Heidelberg (DE); Achim Feurer, Auggen (DE); Ulrich Gradler, Heidelberg (DE); Andreas Goutopoulos, Boston, MA (US); Matthias Schwarz, Geneve (CH)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/667,886

(22) PCT Filed: Nov. 23, 2005
(86) PCT No.: PCT/EP05/12546

§ 371 Date: Aug. 21, 2007

(30) Foreign Application Priority Data
Nov. 24, 2004 (EP) 04 027 918.4

(51) Int. Cl. A61K 31/44; A61P 17/00; A61P 29/00; A61P 3/10; A61P 35/00; A61P 9/00; C07D 213/02

(52) U.S. Cl. 514/340; 514/349; 546/269.4; 546/297

(57) ABSTRACT

The invention provides novel, substituted 4-arylamino pyridone compounds (formula (I)), pharmaceutically acceptable salts, solvates and prodrug compounds thereof, wherein W, R 1, R2, R9, R 10, R 11, R 12, R 13, R 14 and L are as defined in the specification. Such compounds are MEK inhibitors and useful in the treatment of hyperproliferative diseases, such as cancer, restenosis and inflammation. Also disclosed is the use of such compounds in the treatment of hyperproliferative diseases in mammals, especially humans, and pharmaceutical compositions containing such compounds.

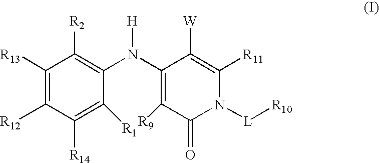

### FIELD OF THE INVENTION

The invention relates to a series of substituted 4-arylamino pyridone derivatives that are useful in the treatment of hyperproliferative diseases, such as cancer and inflammation, in mammals. Also disclosed is the use of such compounds in the treatment of hyperproliferative diseases in mammals, especially humans, and pharmaceutical compositions containing such compounds.

Summary of the related art

The Ras/Raf/MEK/ERK pathway is a central signal transduction pathway, which transmits signals from multiple cell surface receptors to transcription factors in the nucleus which regulate gene expression. This pathway is frequently referred to as the MAP kinase pathway as MAPK stands for mitogen-activated protein kinase indicating that this pathway can be stimulated by mitogens, cytokines and growth factors (Steelman et al., Leukemia 2004, 18, 189-218). Depending upon the stimulus and cell type, this pathway can transmit signals, which result in the prevention or induction of apoptosis or cell cycle progression. The Ras/Raf/MEK/ERK pathway has been shown to play important roles in cell proliferation and the prevention of apoptosis. Aberrant activation of this pathway is commonly observed in malignantly transformed cells. Amplification of ras proto-oncogenes and activating mutations that lead to the expression of constitutively active Ras proteins are observed in approximately 30% of all human cancers (Stirewalt et al., Blood 2001, 97, 3589-95). Mutated, oncogenic forms of Ras are found in 50% of colon and >90% pancreatic cancers as well as many other types of cancers (Kohl et al., Science 1993, 260, 1834-1837). The effects of Ras on proliferation and tumorigenesis have been documented in immortal cell lines (McCubrey et al., Int J Oncol 1995, 7, 295-310). bRaf mutations have been identified in more than 60% of malignant melanoma (Davies, H et al., Nature 2002, 417, 949-954). Given the high level of mutations that have been detected at Ras, this pathway has always been considered a key target for therapeutic intervention (Chang et al., Leukemia 2003, 17,1263-93).

The Ras/Raf/MEK/ERK signaling pathway can exert proliferative or antiproliferative effects through downstream transcription factor targets including NF-κB, CREB, Ets-1, AP-1 and c-Myc. ERKs can directly phosphorylate Ets-1, AP-1 and c-Myc, which lead to their activation. Alternatively, ERKs can phosphorylate and activate a downstream kinase target RSK, which then phosphorylates and activates transcription factors, such as CREB. These transcription factors induce the expression of genes important for cell cycle progression, for example, Cdks, cyclins, growth factors, and apoptosis prevention, for example, antiapoptotic Bcl-2 and cytokines. Overall, treatment of cells with growth factors leads to the activation of ERKs which results in proliferation and, in some cases, differentiation (Lewis et al., Adv. Cancer Res, 1998, 74, 49-139).

MEK proteins are the primary downstream targets of Raf. The MEK family of genes consists of five genes: MEK1, MEK2, MEK3, MEK4 and MEK5. This family of dual-specificity kinases has both serine/threonine and tyrosine kinase activity. The structure of MEK consists of an amino-terminal negative regulatory domain and a carboxy-terminal MAP kinase-binding domain, which is necessary for binding and activation of ERKs. Deletion of the regulatory MEK1 domain results in constitutive MEK1 and ERK activation (Steelman et al., Leukemia 2004, 18, 189-218).

MEK1 is a 393-amino-acid protein with a molecular weight of 44 kDa (Crews et al., Science 1992, 258, 478-80). MEK1 is modestly expressed in embryonic development and is elevated in adult tissue with the highest levels detected in brain tissue. MEK1 requires phosphorylation of S218 and S222 for activation, and substitution of these residues with D or glutamic acid (E) led to an increase in activity and foci formation in NIH3T3 cells (Huang et al., Mol Biol Cell, 1995, 6, 237-45). Constitutive activity of MEK1 in primary cell culture promotes senescence and induces p53 and p16^{INK4a}, and the opposite was observed in immortalized cells or cells lacking either p53 or p16^{INK4a} (Lin et al., Genes Dev, 1998, 12, 3008-3019). Constitutive activity of MEK1 inhibits NF-κB transcription by negatively regulating p38^{MAPK} activity (Carter et al., J Biol Chem 2000, 275, 27858-64). The main physiological substrates of MEK are the members of the ERK (extracellular signal-regulated kinase) or MAPK (mitogen activated protein kinase) family of genes. Aberrant expression of MEK1 has been detected in many different types of cancer, and mutated forms of MEK1 will transform fibroblast, hematopoietic and other cell types.

Constitutive activation of MEK1 results in cellular transformation. It therefore represents a likely target for pharmacological intervention in proliferative and inflammatory diseases (Lee et al., Nature 1994, 372, 739-746; Dudley et al., Proc. Natl. Acad. Sci. U.S.A. 1995, 92, 7686-7689).

Useful inhibitors of MEK have been developed that show potential therapeutic benefit in several studies. For example, small molecule MEK inhibitors have been shown to inhibit human tumor growth in nude mouse xenografts (Yeh, T. et al, Proceedings of the American Association of Cancer Research 2004, 45, Abs 3889 and Lee, P. et al., Proceedings of the American Association of Cancer Research 2004, 45, Abs 3890). MEK inhibitors also entered clinical trials, i.e. ARRY142886 (Wallace, E. et al, Proceedings of the American Association of Cancer Research 2004, 45, Abs 3891), PD-0325901 (Swanton C, Johnston S IDDB MEETING REPORT, Feb. 13-1, 2003) and PD-184352 (Waterhouse et al., Proceedings of the American Society for Clinical Oncology 2003, 22, Abs 816).

Compounds suitable as MEK inhibitors are also disclosed in U.S. Pat. No. 5,525,625; WO 98/43960; WO 99/01421; WO 99/01426; WO 00/41505; WO 00/42002; WO 00/42003; WO 00/41994; WO 00/42022; WO 00/42029; WO 00/68201; WO 01/68619; WO 02/06213; WO03/035626; A2; WO 03/077855; WO03/077914; WO2004/005284; WO2004/056789.

However, PD-184352 was lacking efficacy in clinical phase II trials. Tumors were much less responsive, as no partial responses and only a few patients with stable disease were observed. As a result, the clinical trials of this molecule were suspended (McInnes C IDDB MEETING REPORT 2003). PD-184352 was limited by poor solubility, high metabolic clearance and low bioavailability. This exemplifies the need for novel MEK inhibitors with superior pharmacological properties.

### DESCRIPTION OF THE INVENTION

In view of the foregoing it is the object of the present invention to provide novel MEK inhibitors useful in the treatment of hyperproliferative diseases related to the hyperactivity of MEK as well as diseases modulated by the MEK cascade, such as cancer and inflammation, in mammals with superior pharmacological properties both with respect to their activities as well as their solubility, metabolic clearance and bioavailability characteristics.

As a result, this invention provides novel, substituted 4-arylamino pyridone derivatives and pharmaceutically acceptable salts, solvates or prodrugs thereof, that are MEK inhibitors and useful in the treatment of the above mentioned diseases.

The compounds are defined by Formula (I): 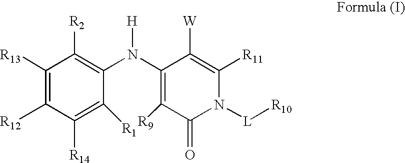
a pharmaceutically acceptable salt, solvate or prodrug thereof, wherein:
R₁, R₂, R₉, R₁₁, R₁₂, R₁₃ and R₁₄ are independently selected from hydrogen, halogen, cyano, nitro, azido, —OR₃, —C(O)R₃, —C(O)OR₃, —NR₄C(O)OR₆, —OC(O)R₃, —NR₄S(O)ⱼR₆, —S(O)ⱼNR₃R₄, —S(O)ⱼNR₄C(O)R₃, —C(O)NR₄S(O)ⱼR₆, S(O)ⱼR₆, —NR₄C(O)R₃, —C(O)NR₃R₄, —NR₅C(O)NR₃R₄, —NR₅C(NCN)NR₃R₄, —NR₃R₄ and C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR₄R₅)ₘ-aryl, aryl, arylalkyl, heteroaryl, heteroarylalkyl, heterocyclyl, heterocyclylalkyl, —O(CR₄R₅)ₘ-aryl, —NR₄(CR₄R₅)ₘ-aryl, —O(CR₄R₅)ₘ-heteroaryl, —NR₄(CR₄R₅)ₘ-heteroaryl, —O(CR₄R₅)ₘ-heterocyclyl, —NR₄(CR₄R5)ₘ-heterocyclyl, and —S(C₁-C₂ alkyl) substituted with 1 to 5 F, where each alkyl, alkenyl, alkynyl, cycloalkyl, aryl, heteroaryl and heterocyclyl is substituted or unsubstituted;
R₁₀ is selected from hydrogen, —OR₃, —C(O)R₃, —C(O)OR₃, —NR₄C(O)OR₆, —OC(O)R₃, —NR₄S(O)ⱼR₆, —S(O)ⱼNR₃R₄, —S(O)ⱼNR₄C(O)R₃, —C(O)NR₄S(O)ⱼR₆, S(O)ⱼR₆, —NR₄C(O)R₃, —C(O)NR₃R₄, —NR₅C(O)NR₃R₄, —NR₅C(NCN)NR₃R₄, —NR₃R₄; —S(O)ⱼ(C₁-C₆ alkyl), —S(O)ⱼ(CR₄R₅)ₘ-aryl, —O(CR₄R₅)ₘ-aryl, —NR₄(CR₄R₅)ₘ-aryl, —O(CR₄R₅)ₘ-heteroaryl, —NR₄(CR₄R₅)ₘ-heteroaryl, -O(CR₄R₅)ₘ-heterocyclyl, —NR₄(CR₄R₅)ₘ-heterocyclyl, and —S(C₁-C₂ alkyl) substituted with 1 to 5 F, where each, aryl, heteroaryl and heterocyclyl is substituted or unsubstituted;
L is selected from C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, heterocyclylalkyl, where each alkyl, alkenyl, alkynyl, cycloalkyl, aryl, heteroaryl and heterocyclyl is unsubstituted or substituted;
or LR₁₀ are together hydrogen;
R₃ is selected from hydrogen, trifluoromethyl, C₁-C₁₀ alkyl, C₂₋₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl is substituted or unsubstituted;
R₄ is selected from hydrogen or C₁-C₆ alkyl whereby alkyl may be substituted or unsubstituted; or
R₃ and R₄ can be taken together with the atom to which they are attached to form a 4 to 10 membered heteroaryl or heterocyclic ring, each of which is substituted or unsubstituted;
R₅ is selected from hydrogen or C₁-C₆ alkyl whereby alkyl may be substituted or unsubstituted; or
R₄ and R₅ can be taken together with the atom to which they are attached to form a 4 to 10 membered carbocyclic, heteroaryl or heterocyclic ring, each of which is substituted or unsubstituted;
R₆ is selected from trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, where each alkyl, cycloalkyl, aryl, heteroaryl and heterocyclyl substituted or unsubstituted;
W is selected from heteroaryl containing 1-4 heteroatoms or heterocyclyl containing 1-4 heteroatoms each of which is unsubstituted or substituted by 1 to 5 substituents ZR₁₅; or W is —C(O)OR₁₅, —C(O)NR₄R₁₅, —C(O)NR₄OR₁₅, —C(O)(C₃-C₁₀ cycloalkyl), —C(O)(C₂-C₁₀ alkyl), —C(O)(aryl), —C(O)(heteroaryl), —C(O)(heterocyclyl), S(O)ⱼNR₄R₁₅, S(O)ⱼNR₄OR₁₅, —S(O)ⱼNR₄C(O)R₁₅, or —C(O)NR₄S(O)ⱼR₆, whereby R₄ and R₁₅ are as defined herein or may form together a 3 to 7 membered ring with 1 or 2 N atoms and optionally an O atom,
Z is a bond, NR₁₆, O, NR₁₆SO₂ or S,
R₁₅ is independently selected from hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl is substituted or unsubstituted;
R₁₆ is selected from hydrogen or C₁-C₁₀ alkyl, or R₁₅ and R₁₆ form together a 4 to 10 membered cyclic ring with 1 or 2 N atoms and optionally an O atom, said ring being substituted or unsubstituted;
m is 0, 1, 2, 3, 4 or 5; and
j is 1 or 2.

In a preferred embodiment, the variants R₁-R₁₆, L, W and Z are defined as above but with the proviso that the following compounds are excluded:

4-(4-Bromo-2-fluoro-phenylamino)-5-chloro-1-methyl-6-oxo-1,6-dihydro-pyridine-3-carboxylic acid cyclopropylmethoxy-amide,

4-(4-Bromo-2-fluoro-phenylamino)-5-fluoro-1-methyl-6-oxo-1,6-dihydro-pyridine-3-carboxylic acid cyclopropylmethoxy-amide,

4-(4-Bromo-2-fluoro-phenylamino)-5-fluoro-1-methyl-6-oxo-1,6-dihydro-pyridine-3-carboxylic acid amide,

1-Benzyl-4-(4-bromo-2-fluoro-phenylamino)-5-chloro-6-oxo-1,6-dihydro-pyridine-3-carboxylic acid(2-hydroxy-ethoxy)-amide,

4-(4-Bromo-2-fluoro-phenylamino)-1,5-dimethyl-6-oxo-1,6-dihydro-pyridine-3-carboxylic acid(2-hydroxy-ethoxy)-amide,

4-(4-Bromo-2-fluoro-phenylamino)-1,5-dimethyl-6-oxo-1,6-dihydro-pyridine-3-carboxylic acid(2-hydroxy-1,1-dimethyl-ethoxy)-amide,

4-(4-Bromo-2-chloro-phenylamino)-1,5-dimethyl-6-oxo-1,6-dihydro-pyridme-3-carboxylic acid(2-hydroxy-1,1-dimethyl-ethoxy)-amide,

4-(4-Bromo-2-chloro-phenylamino)-1,5-dimethyl-6-oxo-1,6-dihydro-pyridine-3-carboxylic acid(2-hydroxy-ethoxy)-amide,

4-(4-Bromo-2-methyl-phenylamino)-1,5-dimethyl-6-oxo-1,6-dihydro-pyridine-3-carboxylic acid(2-hydroxy-ethoxy)-amide,

4-(2,4-Dichloro-phenylamino)-1,5-dimethyl-6-oxo-1,6-dihydro-pyridine-3-carboxylic acid(2-hydroxy-ethoxy)-amide,

4-(4-Bromo-2-fluoro-phenylamino)-5-fluoro-1-methyl-6-oxo-1,6-dihydro-pyridine-3-carboxylic acid ethoxy-amide,

4-(2-Fluoro-4-iodo-phenylamino)-1,5-dimethyl-6-oxo-1,6-dihydro-pyridine-3-carboxylic acid(2-hydroxy-ethoxy)-amide,

5-(5-Amino-[1,3,4]oxadiazol-2-yl)-4-(4-bromo-2-fluoro-phenylamino)-3-fluoro-1-methyl-1H-pyridin-2-one,

4-(2-Fluoro-4-iodo-phenylamino)-1,5-dimethyl-6-oxo-1,6-dihydro-pyridine-3-carboxylic acid(2,3-dihydroxy-propoxy)-amide,

4-(4-Bromo-2-fluoro-phenylamino)-1,5-dimethyl-6-oxo-1,6-dihydro-pyridine-3-carboxylic acid hydroxyamide,

5-Fluoro-4-(2-fluoro-4-methylsulfanyl-phenylamino)-1-methyl-6-oxo-1,6-dihydro-pyridine-3-carboxylic acid ethoxy-amide,

4-(2-Fluoro-4-iodo-phenylamino)-1,5-dimethyl-6-oxo-1,6-dihydro-pyridine-3-carboxylic acid methoxy-amide,

4-(2-Fluoro-4-iodo-phenylamino)-1,5-dimethyl-6-oxo-1,6-dihydro-pyridine-3-carboxylic acid ethoxy-amide,

5-Fluoro-4-(2-fluoro-4-iodo-phenylamino)-1-methyl-6-oxo-1,6-dihydro-pyridine-3-carboxylic acid methoxy-amide,

5-Fluoro-4-(2-fluoro-4-iodo-phenylamino)-1-methyl-6-oxo-1,6-dihydro-pyridine-3-carboxylic acid ethoxy-amide,

5-Fluoro-4-(2-fluoro-4-methylsulfanyl-phenylamino)-1-methyl-6-oxo-1,6-dihydro-pyridine-3-carboxylic acid methoxy-amide,

4-(2-Fluoro-4-methylsulfanyl-phenylamino)-1,5-dimethyl-6-oxo-1,6-dihydro-pyridine-3-carboxylic acid methoxy-amide,

4-(4-Bromo-2-fluoro-phenylamino)-1,5-dimethyl-6-oxo-1,6-dihydro-pyridine-3-carboxylic acid(2-methoxy-ethoxy)-amide,

4-(4-Bromo-2-fluoro-phenylamino)-5-fluoro-1-methyl-6-oxo-1,6-dihydro-pyridine-3-carboxylic acid(2-methoxy-ethoxy)-amide,

4-(4-Bromo-2-fluoro-phenylamino)-1,5-dimethyl-6-oxo-1,6-dihydro-pyridine-3-carboxylic acid methoxy-amide,

4-(4-Bromo-2-fluoro-phenylamino)-1,5-dimethyl-6-oxo-1,6-dihydro-pyridine-3-carboxylic acid ethoxy-amide,

4-(4-Bromo-2-fluoro-phenylamino)-5-fluoro-1-methyl-6-oxo-1,6-dihydro-pyridine-3-carboxylic acid methoxy-amide,

5-(5-Amino-[1,3,4]oxadiazol-2-yl)-4-(4-bromo-2-fluoro-phenylamino)-1,3-dimethyl-1H-pyridin-2-one.

2-(4-Bromo-2-fluoro-phenylamino)-1-methyl-6-oxo-1,6-dihydro-pyridine-3-carboxylic acid cyclopropylmethoxy-amide,

2-(4-Bromo-2-fluoro-phenylamino)-1-methyl-6-oxo-1,6-dihydro-pyridine-3-carboxylic acid(2-hydroxy-ethoxy)-amide,

2-(4-Bromo-2-fluoro-phenylamino)-5-fluoro-1-methyl-6-oxo-1,6-dihydro-pyridine-3-carboxylic acid(2-hydroxy-ethoxy)-amide,

2-(4-Bromo-2-fluoro-phenylamino)-1-ethyl-6-oxo-1,6-dihydro-pyridine-3-carboxylic acid(2-hydroxy-ethoxy)-amide,

2-(2-Fluoro-4-methyl-phenylamino)-1-methyl-6-oxo-1,6-dihydro-pyridine-3-carboxylic acid(2-hydroxy-ethoxy)-amide,

2-(2-Fluoro-4-iodo-phenylamino)-1-methyl-6-oxo-1,6-dihydro-pyridine-3-carboxylic acid methoxy-amide,

(R)-4-(2-Fluoro-4-iodo-phenylamino)-1,5-dimethyl-6-oxo-1,6-dihydro-pyridine-3-carboxylic acid(2,3-dihydroxy-propoxy)-amide,

(R)-4-(4-Bromo-2-fluoro-phenylamino)-1-ethyl-5-fluoro-6-oxo-1,6-dihydro-pyridine-3-carboxylic acid(2-hydroxy-propoxy)-amide,

(S)-4-(4-Bromo-2-fluoro-phenylamino)-1-cyclopropylmethyl-5-methyl-6-oxo-1,6-dihydro-pyridine-3-carboxylic acid(2-hydroxy-propoxy)-amide,

(S)-4-(4-Bromo-2-fluoro-phenylamino)-1-ethyl-5-fluoro-6-oxo-1,6-dihydro-pyridine-3-carboxylic acid(2-hydroxy-propoxy)-amide,

1-Benzyl-4-(4-bromo-2-fluoro-phenylamino)-5-chloro-6-oxo-1,6-dihydro-pyridine-3-carboxylic acid,

1-Benzyl-4-(4-bromo-2-fluoro-phenylamino)-5-chloro-6-oxo-1,6-dihydro-pyridine-3-carboxylic acid amide,

1-Benzyl-4-(4-bromo-2-fluoro-phenylamino)-5-chloro-6-oxo-1,6-dihydro-pyridine-3-carboxylic acid cyclopropylmethoxy-amide

1-Benzyl-4-(4-bromo-2-fluoro-phenylamino)-5-fluoro-6-oxo-1,6-dihydropyridine-3-carboxylic acid,

1-Benzyl-4-(4-bromo-2-fluoro-phenylamino)-5-fluoro-6-oxo-1,6-dihydropyridine-3-carboxylic acid amide,

1-Benzyl-4-(4-bromo-2-fluoro-phenylamino)-5-fluoro-6-oxo-1,6-dihydropyridine-3-carboxylic acid cyclopropylmethoxy-amide,

1-Benzyl-4-(4-bromo-2-fluoro-phenylamino)-5-fluoro6-oxo-1,6-dihydropyridine-3-carboxylic acid(2-hydroxyethoxy)-amide,

1-Benzyl-4-(4-bromo-2-fluoro-phenylamino)-5-fluoro-6-oxo-1,6-dihydropyridine-3-carboxylic acid(2-amino-ethoxy)-amide hydrogen chloride,

1-Benzyl-4-(4-bromo-2-fluoro-phenylamino)-6-oxo-1,6-dihydro-pyridine-3-carboxylic acid amide,

1-Benzyl-4-(4-bromo-2-fluoro-phenylamino)-6-oxo-1,6-dihydro-pyridine-3-carboxylic acid cyclopropylmethoxy-amide,

1-Benzyl-4-(4-bromo-2-fluoro-phenylamino)-6-oxo-1,6-dihydro-pyridine-3-carboxylic acid(2-hydroxy-ethoxy)-amide,

2-(2-Fluoro-2-methyl-phenylamino)-1-methyl-6-oxo-1,6-dihydro-pyridine-3-carboxylic acid(2-hydroxyethoxy)-amide,

2-(4-Bromo-2-fluoro-phenylamino)-6-oxo-1,6-dihydro-pyridine-3-carboxylic acid,

2-(4-Bromo-2-fluoro-phenylamino)-6-oxo-1,6-dihydro-pyridine-3-carboxylic acid cyclopropylmethoxy-amide,

4-(2,4-Dichloro-phenylamino)-1,5-dimethyl-6-oxo-1,6-dihydro-pyridine-3-carboxylic acid,

4-(2,4-Dichloro-phenylamino)-1,5-dimethyl-6-oxo-1,6-dihydro-pyridme-3-carboxylic acid cyclopropylmethoxy-amide,

4-(2,4-Dichloro-phenylamino)-1,5-dimethyl-6-oxo-1,6-dihydro-pyridme-3-carboxylic acid ethoxy-amide,

4-(2-Fluoro-4-methyl-phenylamino)-1,2,5-trimethyl-6-oxo-1,6-dihydro-pyridine-3-carboxylic acid cyclopropylmethoxy-amide,

4-(2-Fluoro-4-methyl-phenylamino)-1,2,5-trimethyl-6-oxo-1,6-dihydro-pyridine-3-carboxylic acid(2-hydroxyethoxy)-amide,

4-(2-Fluoro-4-methyl-phenylamino)-1,3,8-trimethyl-1 H,6H-pyrido[2,3-d]pyridazine-2,5-dione,

4-(2-Fluoro-4-methyl-phenylamino)-1,3-dimethyl-6,7-dihydro-1 H-pyrrolo[3,4-b]pyridine-2,5-dione,

4-(2-Fluoro-4-methyl-phenylamino)-1,3-dimethyl-7,8-dihydro-1H,6H-pyrido[2,3-d]pyridazine-2,5-dione,

4-(4-Bromo-2-chloro-phenylamino)-1,5-dimethyl-6-oxo-1,6-dihydro-pyridine-3-carboxylic acid amide,

4-(4-Bromo-2-chloro-phenylamino)-5-fluoro-1-methyl-6-oxo-1,6-dihydro-pyridine-3-carboxylic acid,

4-(4-Bromo-2-chloro-phenylamino)-5-fluoro-1-methyl-6-oxo-1,6-dihydro-pyridine-3-carboxylic acid cyclopropylmethoxy-amide,

4-(4-Bromo-2-chloro-phenylamino)-5-fluoro-1-methyl-6-oxo-1,6-dihydro-pyridine-3-carboxylic acid amide,

4-(4-Bromo-2-chloro-phenylamino)-5-fluoro-1-methyl-6-oxo-1,6-dihydro-pyridine-3-carboxylic acid(2-hydroxyethoxy)-amide,

4-(4-Bromo-2-fluoro-phenylamino)-1-cyclopropylmethyl-5-methyl-6-oxo-1,6-dihydro-pyridine-3-carboxylic acid,

4-(4-Bromo-2-fluoro-phenylamino)-1-(2-cyclopropylethyl)-5-fluoro-6-oxo-1,6-dihydropyridine-3-carboxylic acid,

4-(4-Bromo-2-fluoro-phenylamino)-1-(2-cyclopropylethyl)-5-fluoro-6-oxo-1,6-dihydropyridine-3-carboxylic acid amide,

4-(4-Bromo-2-fluoro-phenylamino)-1-(2-cyclopropylethyl)-5-fluoro-6-oxo-1,6-dihydropyridine-3-carboxylic acid(2-hydroxethoxy)-amide,

4-(4-Bromo-2-fluoro-phenylamino)-1,3-dimethyl-5-(1H-tetrazol-5-yl)-1H-pyridin-2-one,

4-(4-Bromo-2-fluoro-phenylamino)-1,3-dimethyl-5-(5-oxo-4,5-dihydro-[1,3,4]oxadiazol-2-yl)-1H-pyridin-2-one,

4-(4-Bromo-2-fluoro-phenylamino)-1,3-dimethyl-5-[5-(2-methylaminoethylamino)-[1,3,4]oxadiazol-2-yl)-1H-pyridin-2-one,

4-(4-Bromo-2-fluoro-phenylamino)-1,5-dimethyl-6-oxo-1,6-dihydro-pyridine-3-carboxylic acid,

4-(4-Bromo-2-fluoro-phenylamino)-1,5-dimethyl-6-oxo-1,6-dihydro-pyridine-3-carboxylic acid cyclopropylmethoxy-amide,

4-(4-Bromo-2-fluoro-phenylamino)-1,5-dimethyl-6-oxo-1,6-dihydro-pyridine-3-carboxylic acid amide,

4-(4-Bromo-2-fluoro-phenylamino)-1,5-dimethyl-6-oxo-1,6-dihydro-pyridme-3-carboxylic acid(2-cyanol-ethyl)-amide,

4-(4-Bromo-2-fluoro-phenylamino)-1-cyclohexylmethyl-5-fluoro-6-oxo-1,6-dihydropyridine-3-carboxylic acid,

4-(4-Bromo-2-fluoro-phenylamino)-1-cyclohexylmethyl-5-fluoro-6-oxo-1,6-dihydropyridine-3-carboxylic acid(2-hydroxy-1,1-dimethylethoxy)-amide,

4-(4-Bromo-2-fluoro-phenylamino)-1-cyclohexylmethyl-5-fluoro-6-oxo-1,6-dihydropyridine-3-carboxylic acid cyclopropylmethoxy-amide,

4-(4-Bromo-2-fluoro-phenylamino)-1-cyclopropyl-5-fluoro-6-oxo-1,6-dihydro-pyridine-3-carboxylic acid,

4-(4-Bromo-2-fluoro-phenylamino)-1-cyclopropyl-5-fluoro-6-oxo-1,6-dihydro-pyridine-3-carboxylic acid cyclopropylmethoxy-amide,

4-(4-Bromo-2-fluoro-phenylamino)-1-cyclopropyl-5-fluoro-6-oxo-1,6-dihydro-pyridine-3-carboxylic acid amide,

4-(4-Bromo-2-fluoro-phenylamino)-1-cyclopropyl-5-fluoro-6-oxo-1,6-dihydro-pyridine-3-carboxylic acid(2-hydroxyethoxy)-amide,

4-(4-Bromo-2-fluoro-phenylamino)-1-cyclopropyl-5-fluoro-6-oxo-1,6-dihydro-pyridine-3-carboxylic acid cyclopropylmethoxy-amide,

4-(4-Bromo-2-fluoro-phenylamino)-1-cyclopropylmethyl-5-fluoro-6-oxo-1,6-dihydropyridine-3-carboxylic acid,

4-(4-Bromo-2-fluoro-phenylamino)-1-cyclopropylmethyl-5-fluoro-6-oxo-1,6-dihydropyridine-3-carboxylic acid(2-hydroxy-1,1-dimethylethoxy)-amide,

4-(4-Bromo-2-fluoro-phenylamino)-1-cyclopropylmethyl-5-fluoro-6-oxo-1,6-dihydropyridine-3-carboxylic acid amide,

4-(4-Bromo-2-fluoro-phenylamino)-1-cyclopropylmethyl-5-methyl-6-oxo-1,6-dihydro-pyridine-3-carboxylic acid amide,

4-(4-Bromo-2-fluoro-phenylamino)-1-cyclopropylmethyl-5-methyl-6-oxo-1,6-dihydro-pyridine-3-carboxylic acid(2-hydroxy-1,1-dimethylethoxy)-amide,

4-(4-Bromo-2-fluoro-phenylamino)-1-ethyl-5-fluoro-6-oxo-1,6-dihydro-pyridine-3-carboxylic acid amide,

4-(4-Bromo-2-fluoro-phenylamino)-1-ethyl-5-fluoro-6-oxo-1,6-dihydro-pyridine-3-carboxylic acid(2-hydroxy-1,1-dimethylethoxy)-amide,

4-(4-Bromo-2-fluoro-phenylamino)-1-ethyl-5-fluoro-6-oxo-1,6-dihydro-pyridine-3-carboxylic acid(2-methoxy-ethoxy)-amide,

4-(4-Bromo-2-fluoro-phenylamino)-1-ethyl-5-fluoro-6-oxo-1,6-dihydro-pyridine-3-carboxylic acid methoxy-amide,

4-(4-Bromo-2-fluoro-phenylamino)-1-ethyl-5-fluoro-6-oxo-1,6-dihydro-pyridine-3-carboxylic acid methylamide,

4-(4-Bromo-2-fluoro-phenylamino)-1-ethyl-5-fluoro-6-oxo-_{1,6}-dihydro-pyridine-3-carboxylic acid,

4-(4-Bromo-2-fluoro-phenylamino)-1-ethyl-5-fluoro-6-oxo-1,6-dihydro-pyridine-3-carboxylic acid(2-hydroxyethoxy)-amide,

4-(4-Bromo-2-fluoro-phenylamino)-1-ethyl-5-fluoro-6-oxo-1,6-dihydro-pyridine-3-carboxylic acid(2-hydroxybutoxy)-amide,

4-(4-Bromo-2-fluoro-phenylamino)-1-methyl-6-oxo-1,6-dihydro-pyridine-3-carboxylic acid,

4-(4-Bromo-2-fluoro-phenylamino)-1-methyl-6-oxo-1,6-dihydro-pyridine-3-carboxylic acid cyclopropylmethoxy-amide,

4-(4-Bromo-2-fluoro-phenylamino)-1-methyl-6-oxo-1,6-dihydro-pyridine-3-carboxylic acid(2-hydroxy-ethoxy)-amide,

4-(4-Bromo-2-fluoro-phenylamino)-1-methyl-6-oxo-1,6-dihydro-pyridine-3-carboxylic acid amide,

4-(4-Bromo-2-fluoro-phenylamino)-3-fluoro-1-methyl-5-(5-methyl-4H-[1,2,4]triazol-3-yl)-1H-pyridin-2-one,

4-(4-Bromo-2-fluoro-phenylamino)-3-fluoro-5-[5-(2-hydroxyethylamino)-[1,3,4]oxadiazol-2-yl)-1-methyl-1H-pyridin-2-one,

4-(4-Bromo-2-fluoro-phenylamino)-5-[5-(2-hydroxyethylamino)-[1,3,4]oxadiazol-2-yl)-1,3-dimethyl-1H-pyridin-2-one,

4-(4-Bromo-2-fluoro-phenylamino)-5-chloro-1-methyl-6-oxo-1,6-dihydro-pyridine-3-carboxylic acid amide,

4-(4-Bromo-2-fluoro-phenylamino)-5-chloro-1-methyl-6-oxo-1,6-dihydro-pyridine-3-carboxylic acid(2-hydroxy-ethoxy)-amide,

4-(4-Bromo-2-fluoro-phenylamino)-5-chloro-1-methyl-6-oxo-1,6-dihydro-pyridine-3-carboxylic acid,

4-(4-Bromo-2-fluoro-phenylamino)-5-chloro-1-methyl-6-oxo-1,6-dihydro-pyridine-3-carboxylic acid ethoxy-amide, 4-(4-Bromo-2-fluoro-phenylamino)-5-fluoro-1-(1H-imidazol-4-ylmethyl)-6-oxo-1,6-dihydropyridine-3-carboxylic acid cyclopropylmethoxy-amide,

4-(4-Bromo-2-fluoro-phenylamino)-5-fluoro-1-(1-methyl-1H-imidazol-4-ylmethyl)-6-oxo-1,6-dihydropyridine-3-carboxylic acid,

4-(4-Bromo-2-fluoro-phenylamino)-5-fluoro-1-(1-methyl-1H-imidazol-4-ylmethyl)-6-oxo-1,6-dihydropyridine-3-carboxylic acid cyclopropylmethoxy-amide,

4-(4-Bromo-2-fluoro-phenylamino)-5-fluoro-1-(2-hydroxyethyl)-6-oxo-1,6-dihydropyridine-3-carboxylic acid cyclopropylmethoxy-amide,

4-(4-Bromo-2-fluoro-phenylamino)-5-fluoro-1-(2-methoxyethyl)-6-oxo-1,6-dihydropyridine-3-carboxylic acid,

4-(4-Bromo-2-fluoro-phenylamino)-5-fluoro-1-(2-methoxyethyl)-6-oxo-1,6-dihydropyridine-3-carboxylic acid cyclopropylmethoxy-amide,

4-(4-Bromo-2-fluoro-phenylamino)-5-fluoro-1-(2-morpholin-4-yl-ethyl)-6-oxo-1,6-dihydropyridine-3-carboxylic acid,

4-(4-Bromo-2-fluoro-phenylamino)-5-fluoro-1-(2-morpholin-4-yl-ethyl)-6-oxo-1,6-dihydropyridine-3-carboxylic acid cyclopropylmethoxy-amide,

4-(4-Bromo-2-fluoro-phenylamino)-5-fluoro-1-(6-methylpyridine-2-ylmethyl)-6-oxo-1,6-dihydropyridine-3-carboxylic acid cyclopropylmethoxy-amide,

4-(4-Bromo-2-fluoro-phenylamino)-5-fluoro-1-(6-methylpyridine-2-ylmethyl)-6-oxo-1,6-dihydropyridine-3-carboxylic acid methoxy-amide,

4-(4-Bromo-2-fluoro-phenylamino)-5-fluoro-1-methanesulfonylmethyl-6-oxo-1,6-dihydropyridine-3-carboxylic acid cyclopropylmethoxy-amide,

4-(4-Bromo-2-fluoro-phenylamino)-5-fluoro-1-methyl-6-oxo-1,6-dihydro-pyridine-3-carboxylic acid(2-hydroxy-ethoxy)-amide,

4-(4-Bromo-2-fluoro-phenylamino)-5-fluoro-1-methyl-6-oxo-1,6-dihydro-pyridine-3-carboxylic acid,

4-(4-Bromo-2-fluoro-phenylamino)-5-fluoro-1-methyl-6-oxo-1,6-dihydro-pyridine-3-carboxylic acid tert-butoxy-amide,

4-(4-Bromo-2-fluoro-phenylamino)-5-fluoro-1-methyl-6-oxo-1,6-dihydro-pyridine-3-carboxylic acid cyclopropylmethyl-amide,

4-(4-Bromo-2-fluoro-phenylamino)-5-fluoro-1-methyl-6-oxo-1,6-dihydro-pyridine-3-carboxylic acid methyl-amide,

4-(4-Bromo-2-fluoro-phenylamino)-5-fluoro-1-methyl-6-oxo-1,6-dihydro-pyridine-3-carboxylic acid(2-hydroxy-1,1-dimethylethoxy)-amide,

4-(4-Bromo-2-fluoro-phenylamino)-5-fluoro-1-methyl-6-oxo-1,6-dihydro-pyridine-3-carboxylic acid(2-hydroxy-propoxy)-amide,

4-(4-Bromo-2-fluoro-phenylamino)-5-fluoro-6-oxo-1,6-dihydro-pyridine-3-carboxylic acid hydroxyamide,

4-(4-Bromo-2-fluoro-phenylamino)-5-fluoro-6-oxo-1,6-dihydropyridine-3-carboxylic acid,

4-(4-Bromo-2-fluoro-phenylamino)-5-fluoro-6-oxo-1,6-dihydropyridine-3-carboxylic acid cyclopropylmethoxy-amide,

4-(4-Bromo-2-fluoro-phenylamino)-5-fluoro-6-oxo-1-pyrazin-2-ylmethyl-1,6-dihydropyridine-3-carboxylic acid,

4-(4-Bromo-2-fluoro-phenylamino)-5-fluoro-6-oxo-1-pyrazin-2-ylmethyl-1,6-dihydropyridine-3-carboxylic acid cyclopropylmethoxy-amide,

4-(4-Bromo-2-fluoro-phenylamino)-5-fluoro-6-oxo-1-pyrazin-2-ylmethyl-1,6-dihydropyridine-3-carboxylic acid ethoxy-amide,

4-(4-Bromo-2-fluoro-phenylamino)-5-fluoro-6-oxo-1-pyrazin-2-ylmethyl-1,6-dihydropyridine-3-carboxylic acid propoxy-amide,

4-(4-Bromo-2-fluoro-phenylamino)-5-fluoro-6-oxo-1-pyridazin-3-ylmethyl-1,6-dihydropyridine-3-carboxylic acid cyclopropylmethoxy-amide,

4-(4-Bromo-2-fluoro-phenylamino)-5-fluoro-6-oxo-1-pyridine-2-ylmethyl-1,6-dihydropyridine-3-carboxylic acid,

4-(4-Bromo-2-fluoro-phenylamino)-5-fluoro-6-oxo-1-pyridine-2-ylmethyl-1,6-dihydropyridine-3-carboxylic acid cyclopropylmethoxy-amide,

4-(4-Bromo-2-fluoro-phenylamino)-5-fluoro-6-oxo-1-pyridine-2-ylmethyl-1,6-dihydropyridine-3-carboxylic acid(2-hydroxy-1,1-dimethylethoxy)-amide,

4-(4-Bromo-2-fluoro-phenylamino)-5-fluoro-6-oxo-1-pyridine-2-ylmethyl-1,6-dihydropyridine-3-carboxylic acid(2-hydroxyethoxy)-amide,

4-(4-Bromo-2-fluoro-phenylamino)-5-fluoro-6-oxo-1-pyridine-3-ylmethyl-1,6-dihydropyridine-3-carboxylic acid,

4-(4-Bromo-2-fluoro-phenylamino)-5-fluoro-6-oxo-1-pyridine-3-ylmethyl-1,6-dihydropyridine-3-carboxylic acid cyclopropylmethoxy-amide,

4-(4-Bromo-2-fluoro-phenylamino)-5-fluoro-6-oxo-1-pyridine-3-ylmethyl-1,6-dihydropyridine-3-carboxylic acid propoxy-amide,

4-(4-Bromo-2-fluoro-phenylamino)-5-fluoro-6-oxo-1-pyridine-3-ylmethyl-1,6-dihydropyridine-3-carboxylic acid(3-aminopropoxy)-amide hydrogen chloride,

4-(4-Bromo-2-fluoro-phenylamino)-5-fluoro-6-oxo-1-pyrimidin-4-ylmethyl-1,6-dihydropyridine-3-carboxylic acid cyclopropylmethoxy-amide,

4-(4-Bromo-2-fluoro-phenylamino)-6-oxo-1-phenyl-1,6-dihydro-pyridazine-3-carboxylic acid cyclopropylmethoxy-amide,

4-(4-Bromo-2-fluoro-phenylamino)-6-oxo-1-phenyl-1,6-dihydro-pyridazine-3-carboxylic acid(2-hydroxyethoxy)-amide,

4-(4-Bromo-2-methyl-phenylamino)-1,5-dimethyl-6-oxo-1,6-dihydro-pyridine-3-carboxylic acid,

4-(4-Bromo-2-methyl-phenylamino)-1,5-dimethyl-6-oxo-1,6-dihydro-pyridine-3-carboxylic acid(2-hydroxy-1,1-dimethylethoxy)-amide,

4-(4-Bromo-2-methyl-phenylamino)-1,5-dimethyl-6-oxo-1,6-dihydro-pyridine-3-carboxylic acid cyclopropylmethoxy-amide,

4-(4-Bromo-2-methyl-phenylamino)-5-fluoro-1-methyl-6-oxo-1,6-dihydro-pyridine-3-carboxylic acid ethoxy-amide,

4-(4-Bromo-2-methyl-phenylamino)-5-fluoro-1-methyl-6-oxo-1,6-dihydro-pyridine-3-carboxylic acid cyclopropylmethoxy-amide,

4-(4-Chloro-2-fluoro-phenylamino)-1,5-dimethyl-6-oxo-1,6-dihydro-pyridine-3-carboxylic acid,

4-(4-Chloro-2-fluoro-phenylamino)-1,5-dimethyl-6-oxo-1,6-dihydro-pyridine-3-carboxylic acid(2-hydroxyethoxy)-amide,

4-(4-Chloro-2-fluoro-phenylamino)-1,5-dimethyl-6-oxo-1,6-dihydro-pyridine-3-carboxylic acid cyclopropylmethoxy-amide,

4-(4-Chloro-2-fluoro-phenylamino)-5-fluoro-1-methyl-6-oxo-1,6-dihydro-pyridine-3-carboxylic acid cyclopropylmethoxy-amide,

4-(4-Chloro-2-fluoro-phenylamino)-5-fluoro-1-methyl-6-oxo-1,6-dihydro-pyridine-3-carboxylic acid ethoxy-amide,

4-(4-Chloro-2-fluoro-phenylamino)-5-fluoro-1-methyl-6-oxo-1,6-dihydro-pyridine-3-carboxylic acid amide,

5-(5-Amino-[1,3,4]oxadiazol-2-yl)-4-(4-bromo-2-fluoro-phenylamino)-3-fluoro-1-pyrazin-2-ylmethyl-1H-pyridin-2-one,

5-(5-Amino-[1,3,4]oxadiazol-2-yl)-4-(4-bromo-2-fluoro-phenylamino)-3-fluoro-1,3-dimethyl-1H-pyridin-2-one,

5-(5-Amino-[1,3,4]oxadiazol-2-yl)-4-(4-bromo-2-methyl-phenylamino)-3-fluoro-1-methyl-1H-pyridin-2-one,

5-(5-Amino-[1,3,4]oxadiazol-2-yl)-4-(4-chloro-2-fluoro-phenylamino)-3-fluoro-1-methyl-1H-pyridin-2-one,

5-(5-Amino-[1,3,4]thiadiazol-2-yl)-4-(4-bromo-2-fluoro-phenylamino)-3-fluoro-1-methyl-1H-pyridin-2-one,

5-(5-Amino-4H-[1,3,4]triazol-3-yl)-4-(4-bromo-2-fluoro-phenylamino)-3-fluoro-1-methyl-1H-pyridin-2-one,

5-[5-(2-Amino-ethylamino)-[1,3,4]oxadiazol-2-yl]-4-(4-bromo-2-fluoro-phenylamino)-1,3-dimethyl-1H-pyridin-2-one,

5-[5-(2-Amino-ethylamino)-[1,3,4]oxadiazol-2-yl]-4-(4-bromo-2-fluoro-phenylamino)-3-fluoro-1-methyl-1H-pyridin-2-one hydrogen chloride,

5-Bromo-2-(4-bromo-2-fluoro-phenylamino)-1-methyl-6-oxo-1,6-dihydro-pyridine-3-carboxylic acid(2-hydroxyethoxy)-amide,

5-Bromo-2-(4-bromo-2-fluoro-phenylamino)-1-methyl-6-oxo-1,6-dihydro-pyridine-3-carboxylic acid,

5-Fluoro-4-(2-fluoro-4-methylphenylamino)-1-methyl-6-oxo-1,6-dihydro-pyridine-3-carboxylic acid cyclopropylmethoxy-amide,

5-Fluoro-4-(2-fluoro-4-methylphenylamino)-1-methyl-6-oxo-1,6-dihydro-pyridine-3-carboxylic acid ethoxy-amide

N-[4-(4-Bromo-2-fluoro-phenylamino)-1,5-dimethyl-6-oxo-1,6-dihydro-pyridine-3-carbonyl]-C-phenyl-methanesulfonamide,

N-[4-(4-Bromo-2-fluoro-phenylamino)-1,5-dimethyl-6-oxo-1,6-dihydro-pyridine-3-carbonyl]-methanesulfonamide.

In a further preferred embodiment, the variants R₁-R₁₆, L, W and Z are defined as above on pages 3 to 5 but with the proviso that the compounds according to the following formula are excluded

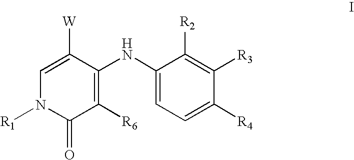

wherein

W is

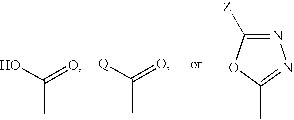

Q is —O—(CH₂)ₖCH₃, —NH₂, —NH[(CH₂)kCH₃], or —NH[O(CH₂)ₖCH₃], wherein the —NH₂ is optionally substituted with between 1 and 2 methyl, and the —(CH₂)ₖCH₃ moieties of the —O—(CH₂)ₖCH₃, —NH[(CH₂)kCH₃], and —NH[O(CH₂)ₖCH₃] groups are optionally substituted with between 1 and 3 substituents independently selected from hydroxy, amino, alkyl and cycloalkyl;

Z is —NH₂ or —NH[(CH₂)kCH₃], wherein the —NH₂ is optionally substituted with between 1 and 2 methyl, and the —(CH₂)ₖCH₃ moiety of the —NH[(CH₂)kCH₃] group is optionally substituted with between 1 and 3 substituents independently selected from hydroxy and amino;

R₁ is hydrogen, C1-6 alkyl, C₂₋₄ alkenyl or —(CH₂)₁₋₃O(CH₂)₁₋₃OCH₃, wherein the C₁₋₆ alkyl is optionally substituted with between 1 and 2 substituents independently selected from hydroxy, —COOH, and cyano;

R₂ is hydrogen, chlorine, fluorine or methyl;

R₃ is hydrogen, chlorine, fluorine, methyl, or CF₃

R₄ is bromine, chlorine, fluorine, iodine, C₁₋₆ alkyl, C₂₋₄ alkenyl, C₂₋₄ alkynyl, C₃₋₆ cycloalkyl, —(CH₂)—C₃₋₆ cycloalkyl, cyano, —O—(C₁₋₄ alkyl), —S—(C₁₋₂ alkyl), —SOCH₃, —SO₂CH₃, —SO₂NR₆R₇, —C≡C—(CH₂)ₙNH₂, —C≡C—(CH₂)ₙNHCH₃, —C≡C—(CH₂)ₙN(CH₃)₂, —C≡C—CH₂OCH₃, —C≡C(CH₂)ₙOH, —C≡C—(CH₂)ₙNH₂, (Z)-CHCHCH₂OCH₃, -(Z)-CHCH—(CH₂)ₙNHCH₃, (Z)-CHCH—(CH₂)ₙN(CH₃)₂, —(CH₂)ₚCO₂R₆, C(O)C₁₋₃ alkyl, C(O)NHCH₃, —(CH₂)ₘNH₂, —(CH₂)ₘNHCH₃, —(CH₂)ₘN(CH₃)₂, —(CH₂)ₘOR₈, —(CH₂)ₚCF₃, —C≡CCF₃, —CH═CHCF₃, —CH₂CHCF₂, —CH═CF₂, —(CF₂)ᵥCF₃, —CH₂(CF₂)ₙCF₃, —(CH₂)ₜCF(CF₃)₂, —CH(CF₃)₂, —CF₂CF(CF₃)₂, or —C(CF₃)₃, wherein the C₁₋₆ alkyl and C₂₋₆ alkynyl are optionally substituted with between 1 and 3 substituents independently selected from hydroxy and alkyl;

R₅ is hydrogen, chlorine, fluorine, or methyl;

R₆ and R₇ are each independently hydrogen, methyl, or ethyl;

k is 0 to 3;

m is 1 to 4;

n is 1 to 2;

p is 0 to 2;

t is 0 to 1;

v is 1 to 5;

and pharmaceutically acceptable salts, C₁₋₆ amides and C₁₋₆ esters thereof.

In a still further preferred embodiment, the variants R₁-R₁₆, L, W and Z are defined as above on pages 3 to 5 but with the proviso that the compounds with the following formula including resolved enantiomers, diastereomers, solvates and pharmaceutically acceptable salts thereof are excluded:

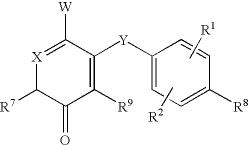

where

X is CR¹⁰;

Y is NH;

R¹, R², R⁸, R⁹ and R¹⁰ are independently hydrogen, halogen, cyano, nitro, azido, —SR¹¹,

—OR³, —C(O)R³, —C(O)OR³, —NR⁴C(O)OR⁶, —OC(O)R³, —NR⁴SO₂R⁶, —SO₂NR³R⁴, —NR⁴C(O)R³, —C(O)NR³R⁴, —NR⁵C(O)NR³R⁴, —NR⁵C(NCN)NR³R⁴, —NR³R⁴, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR⁴R⁵)ₘ-aryl, aryl, arylalkyl, heteroaryl, heteroarylalkyl, heterocyclyl, heterocyclylalkyl, —O(CR⁴R⁵)ₘ-aryl, —NR⁵(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl or —NR⁴(CR⁴R⁵)ₘ-heterocyclyl, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —NR⁴C(O)OR⁶, —NR⁴C(O)R³, —C(O)NR³R⁴, —NR³R⁴, —NR⁵C(O)NR³R⁴, —NR⁵C(NCN)R³R⁴, —OR³, aryl, heteroaryl, arylalkyl, heteroarylalkyl, heterocyclyl, and heterocyclylalkyl,

R⁷ is hydrogen, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, or heterocyclylalkyl, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from —NR¹¹SO₂R¹⁴, —SO₂NR¹¹R¹², —C(O)R¹¹, C(O)OR¹¹, —OC(O)R¹¹, —NR¹¹C(O)OR¹⁴, —NR¹¹C(O)R¹², —C/O)NR¹¹R¹², —SR¹¹, —S(O)R¹⁴, —SO₂R¹⁴, —NR¹¹R₁₂, —NR¹¹C(O)NR¹²R¹³, —NR¹¹C(NCN)NR¹²R¹³, —OR¹¹,

R³is hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, heterocyclylalkyl, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR¹¹SO₂R¹⁴, —SO₂NR¹¹R¹², —C(O)R¹¹, —C(O)OR¹¹, —OC(O)R¹¹, —NR¹¹C(O)OR¹⁴, —NR¹¹C(O)R¹², —C(O)NR¹¹R¹², —SR¹¹, —S(O)R¹⁴, —SO₂R¹⁴, —NR¹¹R¹², —NR¹¹C(O)NR¹²R¹³, —NR¹¹C(NCN)NR¹²R¹³, —OR¹¹, aryl, heteroaryl, arylalkyl, heteroarylalkyl, heterocyclyl, and heterocyclylalkyl, or

R³ and R⁴ together with the atom to which they are attached form a 4 to 10 membered heteroaryl or heterocyclic ring, wherein any of said heteroaryl or heterocyclic rings are optionally substituted with one or more groups independently selected from halogen, cyano, nitro, trifluoromethyl, difluoromethoxy, trifluoromethoxy, azido, —NR¹¹SO₂R¹⁴, —SO₂NR¹¹R¹², —C(O)R¹¹, C(O)OR¹¹, —OC(O)R¹¹, —NR¹¹C(O)OR¹⁴, —NR¹¹C(O)R¹², —C(O)NR¹¹R¹², —SR¹¹, —S(O)R¹⁴, —SO₂R¹⁴, —NR¹¹R¹², —NR¹¹C(O)NR¹²R¹³, —NR¹¹(NCN)NR¹²R¹³, —OR¹¹, aryl, heteroaryl, arylalkyl, heteroarylalkyl, heterocyclyl, and heterocyclylalkyl;

R⁴ and R⁵ independently are hydrogen or C₁-C₆ alkyl, or

R⁴ and R⁵ together with the atom to which they are attached form a 4 to 10-membered carbocyclic, heteroaryl or heterocyclic ring, wherein said alkyl or any of said carbocyclic, heteroaryl and heterocyclic rings are optionally substituted with one or more groups independently selected from halogen, cyano, nitro, trifluoromethyl, difluoromethoxy, trifluoromethoxy, azido, —NR¹¹SO₂R¹⁴, —SO₂NR¹¹R¹², —C(O)R¹¹, C(O)OR¹¹, —OC(O)R¹¹, —NR¹¹C(O)OR¹⁴, —NR¹¹C(O)R¹², —C(O)NR¹¹R¹², —SR¹¹, —S(O)R¹⁴, —SO₂R¹⁴, —NR¹¹R¹², —NR¹¹C(O)NR¹²R¹³, —NR¹¹(NCN)NR¹²R¹³, —OR¹¹, aryl, heteroaryl, arylalkyl, heteroaryl-alkyl, heterocyclyl, and heterocyclylalkyl; R⁶ is trifluoromethyl, C₁-C₁₀ alkyl, C₃-C₁₀ cycloalkyl, aryl, arylalkyl, heteroaryl, heteroarylalkyl, heterocyclyl or heterocyclylalkyl, wherein any of said alkyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR¹¹SO₂R¹⁴, —SO₂NR¹¹R¹², —C(O)R¹¹, C(O)OR¹¹, —OC(O)R¹¹, —NR¹¹C(O)OR¹⁴, —NR¹¹C(O)R¹², —C(O)NR¹¹R¹², —SR¹¹, —S(O)R¹⁴, —SO₂R¹⁴, —NR¹¹R¹², —NR¹¹C(O)NR¹²R¹³, —NR¹¹(NCN)NR¹²R¹³, —OR¹¹, aryl, heteroaryl, arylalkyl, heteroarylalkyl, heterocyclyl, and heterocyclylalkyl;

R¹¹, R¹² and R¹³ independently are hydrogen, lower alkyl, lower alkenyl, aryl and arylalkyl, and

R¹⁴ is lower alkyl, lower alkenyl, aryl and arylalkyl;

or any two of R¹¹, R¹², R¹³ or R¹⁴ together with the atom to which they are attached form a 4 to 10 membered carbocyclic, heteroaryl or heterocyclic ring, wherein any of said alkyl, alkenyl, aryl, arylalkyl carbocyclic rings, heteroaryl rings or heterocyclic rings are optionally substituted with one or more groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl; W is heteroaryl, heterocyclyl, —C(O)OR³, —C(O)NR³R⁴, —C(O)NR⁴OR³, —C(O)NR⁴SO₂R³, —C(O)(C₃-C₁₀ cycloalkyl), —C(O)(C₁-C₁₀ alkyl), —C(O)(aryl), —C(O)(heteroaryl), —C(O)(heterocyclyl), wherein any of said heteroaryl, heterocyclyl, —C(O)OR³, —C(O)NR³R⁴, —C(O)NR⁴OR³, —C(O)NR⁴SO₂R³, —C(O)(C₃-C₁₀ cycloalkyl), —C(O)(C₁-C₁₀ alkyl),

—C(O)(aryl), —C(O)(heteroaryl), —C(O)(heterocyclyl) are optionally substituted with one or more groups independently selected from halogen, cyano, nitro, azido, —NR³R⁴, —OR³, C₁-C₁₀ alkyl, C₂-C₁₀ alkenyl, C₂-C₁₀ alkynyl, cycloalkyl and heterocycloalkyl, wherein any of said C₁-C₁₀ alkyl, C₂-C₁₀ alkenyl, C₂-C₁₀ alkynyl, cycloalkyl and heterocycloalkyl are optionally substituted with 1 or more groups independently selected from —NR³R⁴ and

—OR³;

m is 0, 1, 2, 3, 4 or 5; and

j is 1 or 2.

In more preferred embodiments, the variants have the following meanings:

R₁ is as defined above, preferably hydrogen, halo, C₁-C₄ alkyl, C₃-C₄ cycloalkyl, C₂-C₄ alkenyl, C₂-C₄ alkynyl, cyano, nitro, OR₃ or NR₃R₄; more preferably hydrogen, halo or C₁-C₄ alkyl, still more preferably hydrogen or halo, most preferably hydrogen or F. In one embodiment, R₁ is hydrogen.

In a further embodiment, R₁ is —S(O)ⱼNR₄C(O)R₃, —C(O)NR₄S(O)ⱼR₆, or S(O)ⱼR₆.

R₂ is as defined above, preferably hydrogen, halo, C₁-C₄ alkyl, C₃-C₄ cycloalkyl, C₂-C₄ alkenyl, C₂-C₄ alkynyl, cyano, nitro, OR₃ or NR₃R₄; more preferably hydrogen, halo or C₁-C₂ alkyl, still more preferably halo or methyl, most preferably Cl, F or methyl. In one embodiment, R₂ is methyl. In another embodiment, methyl is preferably further substituted by 1, 2 or 3 fluorines, preferably 3 fluorines. Most preferably, R₂ is F.

In still another embodiment, R₂ is —S(O)ⱼNR₄C(O)R₃, —C(O)NR₄S(O)ⱼR₆, or S(O)ⱼR₆.

R₉ is as defined above, preferably hydrogen, halo, C₁-C₄ alkyl, C₃-C₄ cycloalkyl, C₂-C₄ alkenyl, C₂-C₄ alkynyl, cyano, nitro, OR₃ or NR₃R₄; more preferably hydrogen, halo or C₁-C₄ alkyl, still more preferably hydrogen, methyl or halo, most preferably hydrogen, methyl, Cl or F. In one embodiment, R₉ is hydrogen.

In another embodiment, R₉ is —S(O)ⱼNR₄C(O)R₃, —C(O)NR₄S(O)ⱼR₆, or S(O)ⱼR₆.

R₁₀ is as defined above, preferably hydrogen, —OR₃, —C(O)R₃, —C(O)OR₃, —NR₄C(O)OR₆, —OC(O)R₃, —NR₄S(O)₂R₆, —S(O)₂NR₃R₄, S(O)₂R₆, —NR₄C(O)R₃, —C(O)NR₃R₄, —NR₅C(O)NR₃R₄, —NR₃R₄, more preferably hydrogen, —OR₃, —NR₄C(O)R₃, —C(O)NR₃R₄, —NR₃R₄, still more preferably hydrogen, —OR₃, —NR₃R₄, most preferably hydrogen. In preferred embodiments R₃ and R₄ are independently C₁-C₆ alkyl, more preferably C₁-C₄ alkyl, optionally substituted by 1 or 2 alkyl amino, dialkyl amino, amino, O-alkyl, hydroxy, or R₃ and R₄ form together a cyclic ring with 1 or 2 N atoms and optionally an O atom, said ring being optionally substituted by 1 or 2 alkyl amino, amino, hydroxy or O-alkyl.

In a further embodiment, R₁₀ is —S(O)ⱼNR₄C(O)R₃, —C(O)NR₄S(O)ⱼR₆, —S(O)ⱼ(C₁-C₆ alkyl), —S(O)ⱼ(CR₄R₅)ₘ-aryl, —O(CR₄R₅)ₘ-aryl, —NR₄(CR₄R₅)ₘ-aryl, —O(CR₄R₅)ₘ-heteroaryl, —NR₄(CR₄R₅)ₘ-heteroaryl, —O(CR₄R₅)ₘ-heterocyclyl, or —NR₄(CR₄R₅)ₘ-heterocyclyl, where each aryl, heteroaryl and heterocyclyl is substituted or unsubstituted.

L is as defined above, preferably C₁-C₆ alkyl, C₂-C₆ alkenyl, C₂-C₆ alkynyl, C₃-C₆ cycloalkyl, C₃-C₁₀ cycloalkylalkyl, arylalkyl, heteroarylalkyl, heterocyclyl, heterocyclylalkyl, more preferably C₁-C₅ alkyl, most preferably methylene, ethylene, n-propylene or n-butylene. In one embodiment, L is ethylene, n-propylene or n-butylene. In the definition of L all moieties are divalent so that L serves as a linker between the nitrogen atom and R₁₀.

In one preferred embodiment, LR₁₀ are together methyl.

In another preferred embodiment, LR₁₀ are together hydrogen.

R₁₁ is as defined above, preferably hydrogen, halo, C₁-C₄ alkyl, C₃-C₄ cycloalkyl, C₂-C₄ alkenyl, C₂-C₄ alkynyl, cyano, nitro, OR₃ or NR₃R₄; more preferably hydrogen, halo or C₁-C₄ alkyl or O—C₁-C₄ alkyl, still more preferably hydrogen, methyl, O-methyl or halo, most preferably hydrogen, methyl, Cl, Br or F. In one embodiment, R₁₁ is hydrogen. In another embodiment, R₁₁ is methyl. In yet another embodiment, methyl is preferably further substituted by 1, 2 or 3 fluorines, preferably 3 fluorines.

In still another embodiment, R₁₁ is —S(O)ⱼNR₄C(O)R₃, —C(O)NR₄S(O)ⱼR₆, or S(O)ⱼR₆.

R₁₂ is as defined above, preferably hydrogen, halo, C₁-C₁₀ alkyl, C₃-C₁₀ cycloalkyl, C₂-C₁₀ alkenyl, C₂-C₁₀ alkynyl, cyano, nitro, azido; NR₄SO₂R₆; SO₂NR₃R₄; SO₂R₆; C(O)NR₃R₄; C(O)OR₃; OR₃, NR₃R₄ or —S(C₁-C₂ alkyl) substituted with 1 to 5 F, more preferably hydrogen, halo, nitro, C₁-C₄ alkyl, O—C₁-C₄ alkyl, SCF₃, SCHF₂, SCH₂F, SO₂NR₃R₄ or C(O)NR₃R₄, still more preferably hydrogen, F, Cl, Br, I, nitro, methyl, ethyl, n-propyl, i-propyl, cyclopropyl, O-methyl, SCF₃, SCHF₂, SCH₂F, SO₂NR₃R₄ or C(O)NR₃R₄, most preferably hydrogen I, Cl, Br, SCF₃, SCHF₂, SCH₂F, methyl or O-methyl. In one embodiment R₁₂ is hydrogen. In another embodiment, R₁₂ is methyl, SCF₃, SCHF₂, SCH₂F or O-methyl, wherein methyl or O-methyl is preferably unsubstituted or further substituted by 1, 2 or 3 fluorines, preferably 2 or 3 fluorines. In preferred embodiments of R₁₂, R₃ and R₄ are independently C₁-C₆ alkyl, more preferably C₁-C₄ alkyl, optionally substituted by 1 or 2 alkyl amino, dialkyl amino, amino, O-alkyl, hydroxy, or R₃ and R₄ form together a cyclic ring with 1 or 2 N atoms and optionally an O atom, said ring being optionally substituted by 1 or 2 alkyl amino, amino, hydroxy or O-alkyl. Most preferably, R₁₂ is Br or I.

In still another embodiment, R₁₂ is —S(O)ⱼNR₄C(O)R₃, —C(O)NR₄S(O)ⱼR₆, or S(O)ⱼR₆. Within this embodiment it is preferred that R₆ is C₂-C₁₀ alkyl. R₁₃ is as defined above, preferably hydrogen, halo, C₁-C₄ alkyl, C₃-C₄ cycloalkyl, C₂-C₄ alkenyl or C₂-C₄ alkynyl, more preferably hydrogen, F, Cl or methyl, most preferably hydrogen or F. In one embodiment, R₁₃ is hydrogen.

In another embodiment, R₁₃ is —S(O)ⱼNR₄C(O)R₃, —C(O)NR₄S(O)ⱼR₆, or S(O)ⱼR₆.

R₁₄ is as defined above, preferably hydrogen, halo, C₁-C₄ alkyl, C₃-C₄ cycloalkyl, C₂-C₄ alkenyl or C2-C4 alkynyl, more preferably hydrogen, F, Cl or methyl, most preferably hydrogen or F. In one embodiment, R₁₄ is hydrogen.

In a further embodiment, R₁₄ is —S(O)NR₄C(O)R₃, —C(O)NR₄S(O)ⱼR₆, or S(O)ⱼR₆.

In a particular preferred embodiment, at least one of R₁, R₂, R₉, R₁₁, R₁₂, R₁₃ and R₁₄ is selected from —S(O)ⱼNR₄C(O)R₃, —C(O)NR₄S(O)ⱼR₆, and S(O)ⱼR₆.

As set forth above, the variants of each of R₁, R₂, R₉ to R₁₄ and L may be substituted. In this case they can be substituted with 1 to 5, preferably 1 to 3, more preferably 1 or 2 groups independently selected from oxo, halogen, cyano, nitro, CF₃, CHF₂, CH₂F, OCF₃, OCHF₂, OCH₂F, SCF₃, SCHF₂, SCH₂F, azido, NR₄SO₂R₆, SO₂NR₃R₄, C(O)R₃, C(O)OR₃, OC(O)R₃, NR₄C(O)OR₆, NR₄C(O)R₃, C(O)NR₃R₄, NR₃R₄, NR₅C(O)NR₃R₄, NR₅C(NCN)NR₃R₄, OR₃, aryl, heteroaryl, arylalkyl, heteroarylalkyl, heterocyclyl, and heterocyclylalkyl, preferably oxo, halogen, cyano, nitro, CF₃, CHF₂, CH₂F, OCF₃, OCHF₂, OCH₂F, SCF₃, SCHF₂, SCH₂F, azido, NR₄SO₂R₆, SO₂NR₃R₄, C(O)R₃, C(O)OR₃, OC(O)R₃, OR₃, more preferably oxo, halogen, cyano, nitro, trifluoromethyl, difluoromethoxy, trifluoromethoxy or azido, most preferably halogen, cyano, nitro, CF₃, CHF₂, CH₂F, OCF₃, OCHF₂, OCH₂F, SCF₃, SCHF₂, SCH₂F, OH, O-methyl, NH₂ or N(methyl)₂. When it is described that alkyl, alkenyl, alkynyl, cycloalkyl, aryl, heteroaryl and heterocyclyl are substituted, this refers to any alkyl, alkenyl, alkynyl, cycloalkyl, aryl, heteroaryl and heterocyclyl as a group or sub-structure such as in cycloalkylalkyl, arylalkyl, heteroarylalkyl, heterocyclylalkyl.

R₃ is as defined above, preferably hydrogen, trifluoromethyl, C₁-C₄ alkyl, C₂-C₄ alkenyl, C₂-C₄ alkynyl, C₃-C₆ cycloalkyl, C₃-C₆ cycloalkylalkyl, more preferably hydrogen or C₁-C₄ alkyl most preferably hydrogen, methyl or ethyl.

R₄ is as defined above, preferably hydrogen or C₁-C₄ alkyl, more preferably hydrogen, methyl or ethyl.

In one preferred embodiment, R₃ and R₄ can be taken together with the atom to which they are attached to form a 4 to 7, preferably 5 or 6, membered heteroaryl or heterocyclic ring.

R₅ is as defined above, preferably hydrogen or C₁-C₄ alkyl, more preferably hydrogen, methyl or ethyl.

In one embodiment, R₄ and R₅ can be taken together with the atom to which they are attached to form a 4 to 7, preferably 5 or 6, membered carbocyclic, heteroaryl or heterocyclic ring.

R₆ is as defined above, preferably trifluoromethyl, C₁-C₄ alkyl, C₂-C₄ alkenyl, C₂-C₄ alkynyl, C₃-C₆ cycloalkyl, C₃-C₆ cycloalkylalkyl, more preferably C₁-C₄ alkyl, most preferably methyl or ethyl.

As set forth above, the variants of each of R₃, R₄, R₅, R₆ or the rings formed by R₃ and R₄ and R₄ and R₅ may be substituted. In this case they can be substituted with 1 to 5, preferably 1 to 3, more preferably 1 or 2 groups independently selected from oxo, halogen, cyano, nitro, CF₃, CHF₂, CH₂F, OCF₃, OCHF₂, OCH₂F, azido, NR′SO₂R″″, SO₂NR″, C(O)R′, C(O)OR′, OC(O)R′, NR′C(O)OR″″, NR′C(O)R″, C(O)NR′R″, SR″″, S(O)R″″, SO₂R′, NR′R″, NR′C(O)NR″R′″, NR′C(NCN)NR″R′″, OR′, aryl, heteroaryl, arylalkyl, heteroarylalkyl, heterocyclyl, and heterocyclylalkyl, preferably oxo, halogen, cyano, nitro, CF₃, CHF₂, CH₂F, OCF₃, OCHF₂, OCH₂F, azido, NR′SO₂R″″, SO₂NR″, C(O)R′, C(O)OR′, OC(O)R′, NR′C(O)OR″″, NR′C(O)R″, C(O)NR′R″, SR″″, S(O)R″″, SO₂R′, NR′R″, NR′C(O)NR″R′″, NR′C(NCN)NR″R′″ or OR′, more preferably oxo, halogen, cyano, nitro, CF₃, CHF₂, CH₂F, OCF₃, OCHF₂, OCH₂F, azido, SR″″, S(O)R″″, SO₂R′, NR′R″ or OR′. In one embodiment, R₃ is preferably oxo, halogen, nitro, trifluoromethyl, OH, O-methyl, NH₂ or N(methyl)₂. When it is described that alkyl, alkenyl, alkynyl, cycloalkyl, aryl, heteroaryl and heterocyclyl are substituted, this refers to any alkyl, alkenyl, alkynyl, cycloalkyl, aryl, heteroaryl and heterocyclyl as a group or sub-structure such as in cycloalkylalkyl, arylalkyl, heteroarylalkyl, heterocyclylalkyl.

R′ is selected from hydrogen, C₁-C₄ alkyl, C₂-C₄ alkenyl, aryl and arylalkyl, preferably hydrogen or C₁-C₄ alkyl, more preferably hydrogen or methyl.

R″ is selected from hydrogen, C₁-C₄ alkyl, C₂-C₄ alkenyl, aryl and arylalkyl, preferably hydrogen or C₁-C₄ alkyl, more preferably hydrogen or methyl.

R′″ is selected from hydrogen, C₁-C₄ alkyl, C₂-C₄ alkenyl, aryl and arylalkyl, preferably hydrogen or C₁-C₄ alkyl, more preferably hydrogen or methyl.

R″″ is selected from C₁-C₄ alkyl, C₁-C₄ alkenyl, aryl and arylalkyl, preferably C₁-C₄ alkyl, more preferably methyl.

Alternatively, any two of R′, R″, R′″ or R″″ can be taken together with the atom to which they are attached to form a 4 to 10 membered carbocyclic, heteroaryl or heterocyclic ring, each of which is optionally substituted with one to three groups independently selected from halogen, cyano; nitro, CF₃, CHF₂, CH₂F, OCF₃, OCHF₂, OCH₂F, azido, aryl, heteroaryl, arylalkyl, heteroarylalkyl, heterocyclyl, and heterocyclylalkyl, preferably halogen, cyano; nitro, trifluoromethyl, difluoromethoxy, trifluoromethoxy and azido.

W is as defined above, preferably heteroaryl containing 1, 2 or 3 heteroatoms, or heterocyclyl containing 1, 2,or 3 heteroatoms, more preferably heteroaryl, each of which is unsubstituted or substituted by 1 to 5, preferably 1 to 3, more preferably 1, substituents ZR₁₅, or W is —C(O)OR₁₅, —C(O)NR₄R₁₅, —C(O)NR₄OR₁₅, —C(O)(C₃-C₁₀ cycloalkyl), —C(O)(C₂-C₁₀ alkyl), —S(O)ⱼNR₄C(O)R₁₅, —C(O)NR₄S(O)ⱼR₆, S(O)ⱼNR₄R₁₅ or S(O)ⱼNR₄OR₁₅, more preferably W is heteroaryl containing 1, 2, or 3, specifically 2 or 3 N atoms, C(O)NR₄OR₁₅ or S(O)₂NR₄OR₁₅. R₄ and R₁₅ are as defined herein or may form together a 3 to 7 membered ring with 1 or 2 N atoms and optionally an O atom. When W is heteroaryl, it is preferably 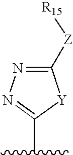
where Z and R₁₅ are as defined above, preferably Z is a bond, NR₁₆, NR₁₆SO₂ or O, more preferably NR₁₆, wherein R₁₆ is as defined above, preferably hydrogen or C₁-C₄ alkyl, more preferably hydrogen. R₁₅ is preferably selected from hydrogen, C₁-C₄ alkyl, C₁-C₄ alkenyl, C₄-C₈ cycloalkylalkyl, each may contain 1 N atom optionally an O atom, where alkyl, alkenyl or cycloalkylalkyl may be further substituted by 1 or 2 of OH, O—C₁-C₄ alkyl or NR′R″, where R′ and R″ are independently hydrogen or C₁-C₄ alkyl where R′ and R″ may form a 3 to 7 membered ring with 1 or 2 N atoms and optionally an O atom. Alternatively, R₁₆ and R₁₅ may form together a 4 to 10 membered cyclic ring with 1 or 2 N atoms and optionally an O atom, said ring being optionally substituted by 1 or 2 alkyl amino, amino, hydroxy or O-alkyl. More preferably R₁₅ is C₁-C₄ alkyl or C₁-C₄ alkenyl optionally substituted with 1 substituent OH, O-Me, NH₂, N(methyl)₂ or N(ethyl)₂.

Y is O or NR′, preferably O.

Alternatively, W is preferably —C(O)OR₁₅, —C(O)NR₄R₁₅, —C(O)NR₄OR₁₅, S(O)ⱼNR₄R₁₅ or S(O)ⱼNR₄OR₁₅, more preferably —C(O)NR₄OR₁₅ or S(O)₂NR₄OR₁₅. In these cases R₁₅ is preferably as defined below.

In yet another embodiment, W is —S(O)ⱼNR₄C(O)R₁₅, whereby R₄ and R₁₅ are as defined herein or may form together a 3 to 7 membered ring with 1 or 2 N atoms and optionally an O atom,

Z is as defined above, preferably a bond, NR₁₆, NR₁₆SO₂ or O, more preferably NR₁₆.

In another embodiment, Z is S.

R₁₅ is as defined above, preferably hydrogen, C₁-C₄ alkyl, C₁-C₄ alkenyl, C₄-C₆ cycloalkylalkyl, more preferably C₁-C₄ alkyl or C₁-C₄ alkenyl, yet more preferably C₁-C₄ alkyl. Alkyl, alkenyl, cycloalkyl, alkynyl, aryl, heteroaryl or heterocyclyl may be further substituted with 1 to 5, preferably 1, 2 or 3, more preferably 1 or 2, substituents selected from OR₃ or NR′R″ wherein R₃ is selected from hydrogen, C₁-C₄ alkyl or C₁-C₄ alkenyl, C₄-C₆ cycloalkylalkyl, more preferably hydrogen, methyl or ethyl, and where R′ and R″ are independently hydrogen or C₁-C₄ alkyl, or R′ and R″ may form a 3 to 7 membered ring with 1 or 2 N atoms and optionally an O atom, more preferably R′ and R″ are independently hydrogen, methyl or ethyl, still more preferably both R′ and R″ are methyl. Yet more preferably, R₁₅ may be substituted by 1 or2 of OH, O—C₁-C₄ alkyl or NR′R″.

Most preferably, R₁₅ is C₁-C₄ alkyl or C₁-C₄ alkenyl optionally substituted with 1 substituent OH, O-Me, NH₂, N(methyl)₂ or N(ethyl)₂.

Regarding R₁₅, when it is described that alkyl, alkenyl, alkynyl, cycloalkyl, aryl, heteroaryl and heterocyclyl are substituted, this refers to any alkyl, alkenyl, alkynyl, cycloalkyl, aryl, heteroaryl and heterocyclyl as a group or sub-structure such as in cycloalkylalkyl, arylalkyl, heteroarylalkyl, heterocyclylalkyl.

R₁₆ is as defined above, preferably hydrogen or C₁-C₄ alkyl, more preferably hydrogen.

Alternatively, R₁₆ and R₁₅ may form together a 4 to 10, preferably 5 to 6, membered cyclic ring with 1 or 2 N atoms and optionally an O atom, said ring being optionally substituted by 1 or 2 alkyl amino, amino, hydroxy or O-alkyl.

m is as defined above, preferably 0, 1, 2 or 3, more preferably 0, 1 or 2, most preferably 1.

j is as defined above, preferably 2.

In the above, any of the preferred definitions for each variant can be combined with the preferred definition of the other variants.

The combinations as set forth in the claims are particularly preferred.

In the above and the following, the employed terms have independently the meaning as described below:

Aryl is an aromatic mono- or polycyclic moiety with preferably 6 to 20 carbon atoms which is preferably selected from phenyl, biphenyl, naphthyl, tetrahydronaphthyl, fluorenyl, indenyl or phenanthrenyl, more preferably phenyl or naphthyl.

Heteroaryl is an aromatic moiety having 6 to 20 carbon atoms with at least one ring containing a heteroatom selected from O, N and/or S, or heteroaryl is an aromatic ring containing at least one heteroatom selected from O, N and/or S and 1 to 6 carbon atoms. Preferably, heteroaryl contains 1 to 4, more preferably 1, 2 or 3 heteroatoms selected from O and/or N and is preferably selected from pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Spiro moieties are also included within the scope of this definition. Preferred heteroaryl include pyridinyl, imidazolyl, pyrimidinyl, pyrazolyl, triazolyl, pyrazinyl, tetrazolyl, isoxazolyl, oxazolyl, isothiazolyl, oxadiazolyl, triazolyl. Heteroaryl groups are optionally mono-, di-, or trisubstituted with, e.g., halogen, lower alkyl, lower alkoxy, haloalkyl, aryl, heteroaryl, and hydroxy.

Heterocyclyl is a saturated or unsaturated ring containing at least one heteroatom selected from O, N and/or S and 1 to 6 carbon atoms. Preferably, heterocyclyl contains 1 to 4, more preferably 1, 2 or 3 heteroatoms selected from O and/or N and is preferably selected from pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, azetidin-2-one-1-yl, pyrrolidin-2-one-1-yl, piperid-2-one-1-yl, azepan-2-one-1-yl, 3-azabicyco[3.1.0]hexanyl, 3-azabicyclo[4.1.0]heptanyl, azabicyclo[2.2.2]hexanyl, 3H-indolyl and quinolizinyl. Spiromoieties are also included within the scope of this definition.

Carbocyclyl is a monocyclic or polycyclic ring system of 3 to 20 carbon atoms which may be saturated, unsaturated or aromatic.

Alkyl is a saturated hydrocarbon moiety, namely straight chain or branched alkyl having 1 to 10, preferably 1 to 8 carbon atoms, more preferably 1 to 4 carbon atoms, such as methyl, ethyl, n-propyl, isopropyl, n-butyl, isobutyl, tert-butyl, n-pentyl, isopentyl, neopentyl, hexyl or heptyl.

Cycloalkyl is an alkyl ring having 3 to 10, preferably 3 to 8 carbon atoms, more preferably 3 to 6 carbon atoms, such as cyclopropyl, cyclobutyl, cyclopentyl, cyclohexyl, cycloheptyl or cyclooctyl.

Alkenyl is an unsaturated hydrocarbon moiety with one or more double bonds, preferably one double bond, namely straight chain or branched alkenyl having 1 to 10, preferably 2 to 8 carbon atoms, more preferably 2 to 4 atoms, such as vinyl, allyl, methallyl, buten-2-yl, buten-3-yl, penten-2-yl, penten-3-yl, penten-4-yl, 3-methyl-but-3-enyl, 2-methyl-but-3-enyl, 1-methyl-but-3-enyl, hexenyl or heptenyl.

Alkynyl is an unsaturated hydrocarbon moiety with one or more triple bonds, preferably one triple bond, namely straight chain or branched alkynyl having 1 to 10, preferably 2 to 8 carbon atoms, more preferably 2 to 4 atoms, such as ethynyl, propynyl, butyn-2-yl, butyn-3-yl, pentyn-2-yl, pentyn-3-yl, pentyn-4-yl, 2-methyl-but-3-ynyl, 1-methyl-but-3-ynyl, hexynyl or heptynyl.

Halo or halogen is a halogen atom preferably selected from F, Cl, Br and I, preferably F, Cl and Br.

In the definitions cycloalkylalkyl, arylalkyl, heteroarylalkyl and heterocyclylalkyl it is contemplated that cycloalkyl, aryl, heteroaryl and heterocyclyl are bonded via an alkylene moiety. This alkylene moiety may be a straight chain or branched chain group. Said alkylene moiety preferably has 1 to 6 carbon atoms. Examples thereof include methylene, ethylene, n-propylene, n-butylene, n-pentylene, n-hexylene, iso-propylene, sec.-butylene, tert.-butylene, 1,1-dimethyl propylene, 1,2-dimethyl propylene, 2,2-dimethyl propylene, 1,1-dimethyl butylene, 1,2-dimethyl butylene, 1,3-dimethyl butylene, 2,2-dimethyl butylene, 2,3-dimethyl butylene, 3,3-dimethyl butylene, 1-ethyl butylene, 2-ethyl butylene, 3-ethyl butylene, 1-n-propyl propylene, 2-n-propyl propylene, 1-iso-propyl propylene, 2-iso-propyl propylene, 1-methyl pentylene, 2-methyl pentylene, 3-methyl pentylene and 4-methyl pentylene. More preferably, said alkylene moiety has 1 to 3 carbon atoms, such as methylene, ethylene, n-propylene and iso-propylene. Most preferred is methylene.

Preferred embodiments of the compounds according to present invention are shown in scheme 1.

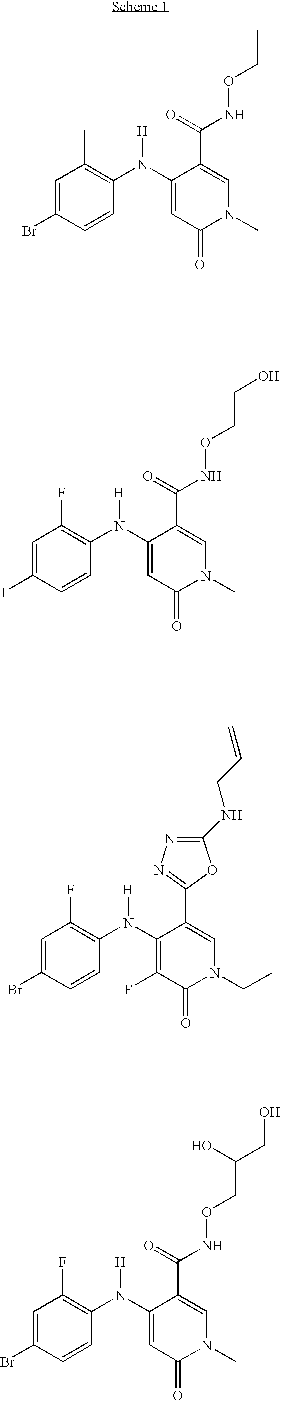

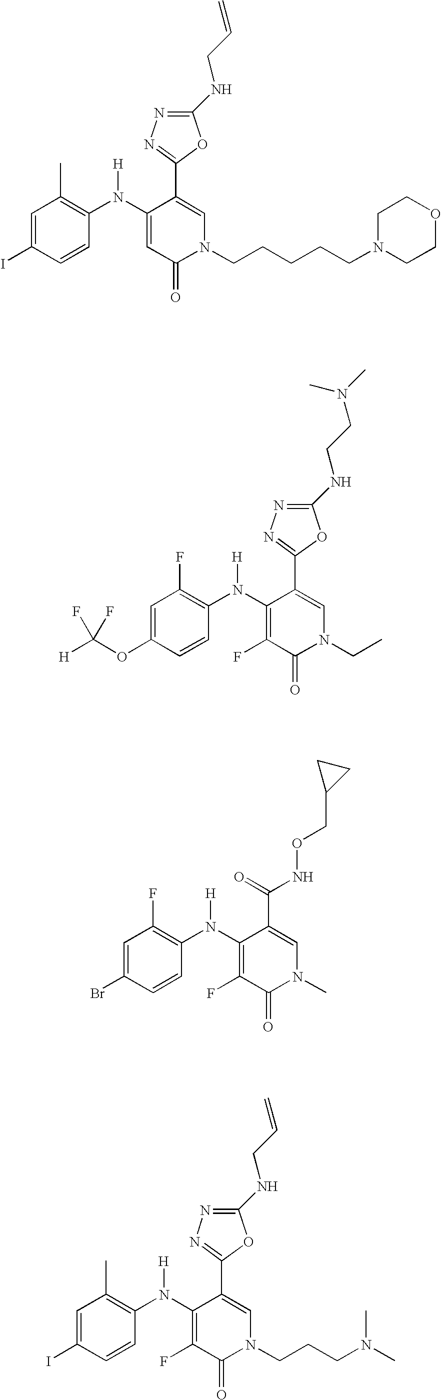

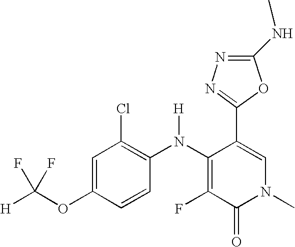

The compounds of the present invention can be in the form of a prodrug compound. “Prodrug compound” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of the prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods. Other examples of the prodrug are compounds, wherein the carboxylate in a compound of the present invention is for example converted into an alkyl-, aryl-, choline-, amino, acyloxymethylester, linolenoyl-ester.

Metabolites of compounds of the present invention are also within the scope of the present invention.

Where tautomerism, like e.g. keto-enol tautomerism, of compounds of the present invention or their prodrugs may occur, the individual forms, like e.g. the keto and enol form, are claimed separately and together as mixtures in any ratio. Same applies for stereoisomers, like e.g. enantiomers, cis/trans isomers, conformers and the like.

If desired, isomers can be separated by methods well known in the art, e.g. by liquid chromatography. Same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials.

The compounds of the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the of the present invention which contain acidic groups can be present on these groups and can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the present invention which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Furthermore, the present invention provides pharmaceutical compositions comprising a compound of the present invention, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

A pharmaceutical composition of the present invention may additionally comprise one or more other compounds as active ingredients like one or more additional compounds of the present invention, or a prodrug compound or other MEK inhibitors.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In one embodiment, said compounds and pharmaceutical composition are for the treatment of cancer such as brain, lung, squamous cell, bladder, gastic, pancreatic, breast, head, neck, renal, kidney, ovarian, prostate, colorectal, oesohageal, testicular, gynecological or thyroid cancer. In another embodiment, said pharmaceutical composition is for the treatment of a noncancerous hyperproliferative disorder such as benign hyperplasia of the skin (e.g., psoriasis), restenosis, or prostate (e.g. benign prostatic hypertrophy (BPH)).

The invention also relates to a compound or pharmaceutical composition for the treatment of pancreatitis or kidney disease (including proliferative glomerulonephtitis and diabetes induced renal disease) or pain in a mammal which comprises a therapeutically effective amount of a compound of the present invention, or a pharmaceutically acceptable salt, prodrug or hydrate thereof, and a pharmaceutically acceptable carrier. The invention also relates to a compound or pharmaceutical composition for the prevention of blastocyte implantation in a mammal which comprises a therapeutically effective amount of a compound of the present invention, or a pharmaceutically acceptable salt, prodrug or hydrate thereof, and a pharmaceutically acceptable carrier. The invention also relates to a compound or pharmaceutical composition for treating a disease related to vasculogenesis or angiogenesis in a mammal which comprises a therapeutically effective amount of a compound of the present invention, or a pharmaceutically acceptable salt, prodrug or hydrate thereof, and a pharmaceutically acceptable carrier.

In one embodiment, said compound or pharmaceutical composition is for treating a disease selected from the group consisting of tumor angiogenesis, chronic inflammatory disease such as rheumatoid arthritis, inflammatory bowel disease, atherosclerosis, skin diseases such as psoriasis, excema, and sclerodema, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

The invention also relates to of the use for treating a hyperproliferative disorder in a mammal that comprises administering to said mammal a therapeutically effective amount of a compound of the present invention, or a pharmaceutically acceptable salt, prodrug or hydrate thereof. In one embodiment, said use relates to the treatment of cancer such as brain, lung, squamous cell, bladder, gastic, pancreatic, breast, head, neck, renal, kidney, ovarian, prostate, colorectal, oesohageal, testicular, gynecological or thyroid cancer. In another embodiment, said use relates to the treatment of a non-cancerous hyperproliferative disorder such as benign hyperplasia of the skin (e.g., psoriasis), restenosis, or prostate (e.g. benign prostatic hypertrophy (BPH)).

The invention also relates to a use for the treatment of a hyperproliferative disorder in a mammal that comprises administering to said mammal a therapeutically effective amount of a compound of the present invention, or a pharmaceutically acceptable salt, prodrug or hydrate thereof, in combination with an anti-tumor agent selected from the group consisting of mitotic inhibitors, alkylating agents, antimetabolites, intercalating antibiotics, growth factor inhibitors, cell cycle inhibitors, enzyme inhibitors, topoisomerase inhibitors, biological response modifiers, antihormones, angiogenesis inhibitors, and anti-androgens.

The invention also relates to a use of treating pancreatitis or kidney disease or pain in a mammal that comprises administering to said mammal a therapeutically effective amount of a compound of the present invention, or a pharmaceutically acceptable salt, prodrug or hydrate thereof. The invention also relates to a use of preventing blastocyte implantation in a mammal that comprises administering to said mammal a therapeutically effective amount of a compound of the present invention, or a pharmaceutically acceptable salt, prodrug or hydrate thereof.

The invention also relates to a use of treating diseases related to vasculogenesis or angiogenesis in a mammal that comprises administering to said mammal a therapeutically effective amount of a compound of the present invention, or a pharmaceutically acceptable salt, prodrug or hydrate thereof. In one embodiment, said method is for treating a disease selected from the group consisting of tumor angiogenesis, chronic inflammatory disease such as rheumatoid arthritis, atherosclerosis, inflammatory bowel disease, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer. Patients that can be treated with compounds of the present invention, or pharmaceutically acceptable salts, prodrugs and hydrates of said compounds, according to the methods of this invention include, for example, patients that have been diagnosed as having psoriasis, restenosis, atherosclerosis, BPH, lung cancer, bone cancer, CMML, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, testicular, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphona, spinal axis tumors, brain stem gliomas or pituitary adenomas).

This invention also relates to a compound or pharmaceutical composition for inhibiting abnormal cell growth in a mammal which comprises an amount of a compound of the present invention, or a pharmaceutically acceptable salt or solvate or prodrug thereof, in combination with an amount of a chemotherapeutic, wherein the amounts of the compound, salt, solvate, or prodrug, and of the chemotherapeutic are together effective in inhibiting abnormal cell growth. Many chemotherapeutics are presently known in the art. In one embodiment, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. This invention further relates to a method for inhibiting abnormal cell growth in a mammal or treating a hyperproliferative disorder which method comprises administering to the mammal an amount of a compound of the present invention, or a pharmaceutically acceptable salt or solvate or prodrug thereof, in combination with radiation therapy, wherein the amounts of the compound, salt, solvate, or prodrug, is in combination with the radiation therapy effective in inhibiting abnormal cell growth or treating the hyperproliferative disorder in the mammal. Techniques for administering radiation therapy are known in the art, and these techniques can be used in the combination therapy described herein. The administration of the compound of the invention in this combination therapy can be determined as described herein. It is believed that the compounds of the present invention can render abnormal cells more sensitive to treatment with radiation for purposes of killing and/or inhibiting the growth of such cells. Accordingly, this invention further relates to a method for sensitizing abnormal cells in a mammal to treatment with radiation which comprises administering to the mammal an amount of a compound of the present invention or pharmaceutically acceptable salt or solvate or prodrug thereof, which amount is effective is sensitizing abnormal cells to treatment with radiation. The amount of the compound, salt, or solvate in this method can be determined according to the means for ascertaining effective amounts of such compounds described herein. The invention also relates to a method of and to a pharmaceutical composition of inhibiting abnormal cell growth in a mammal which comprises an amount of a compound of the present invention, or a pharmaceutically acceptable salt or solvate thereof, a prodrug thereof, or an isotopically-labeled derivative thereof, and an amount of one or more substances selected from anti-angiogenesis agents, signal transduction inhibitors, and antiproliferative agents.

In practical use, the compounds of the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

When treating or preventing cancer, inflammation or other proliferative diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligram to about 50 milligrams. In the case of a 70 kg adult human, the total daily dose will generally be from about 7 milligrams to about 350 milligrams. This dosage regimen may be adjusted to provide the optimal therapeutic response.

Some abbreviations that may appear in this application are as follows.

Abbreviations

Designation

b Broad peak

Boc tert.-Butyloxycarbonyl

CDI N,N-Carbonyldiimidazole

d Doublet

DCM Dichloromethane

dd double doublet

DIPEA N-Ethyldiisopropylamine

DMF N,N-Dimethylformamide

DMSO Dimethylsulfoxide

EDC 1-(3-Dimethylaminopropyl)-3-ethylcarbodiimide hydrochloride

HPLC High pressure liquid chromatography

LiHMDS. Lithium hexamethyldisilazide

NMR Nuclear Magnetic Resonance

PG Protecting group

PyBroP Bromo-tris-pyrrolidino-phosphonium hexafluorophosphate

PYBOP Benzotriazole-1-yl-oxy-trispyrrolidinophosphonium

hexafluorophosphate

q Quartett

rt Retention time

s Singlet

tert Tertiary-butyl

TFA Trifluoroacetic acid

THF Tetrahydrofurane

TLC Thin Layer Chromatography

The compounds of the present invention can be prepared according to the procedures of the following Schemes and Examples, using appropriate materials and are further exemplified by the following specific examples. Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described above. The amine-free bases corresponding to the isolated salts can be generated by neutralization with a suitable base, such as aqueous sodium hydrogencarbonate, sodium carbonate, sodium hydroxide and potassium hydroxide, and extraction of the liberated amine-free base into an organic solvent, followed by evaporation. The amine-free base, isolated in this manner, can be further converted into another pharmaceutically acceptable salt by dissolution in an organic solvent, followed by addition of the appropriate acid and subsequent evaporation, precipitation or crystallization.

An illustration of the preparation of compounds of the present invention is shown in schemes 2 and 3. Unless otherwise indicated in the schemes, the variables have the same meaning as described above.

The examples presented below are intended to illustrate particular embodiments of the invention.

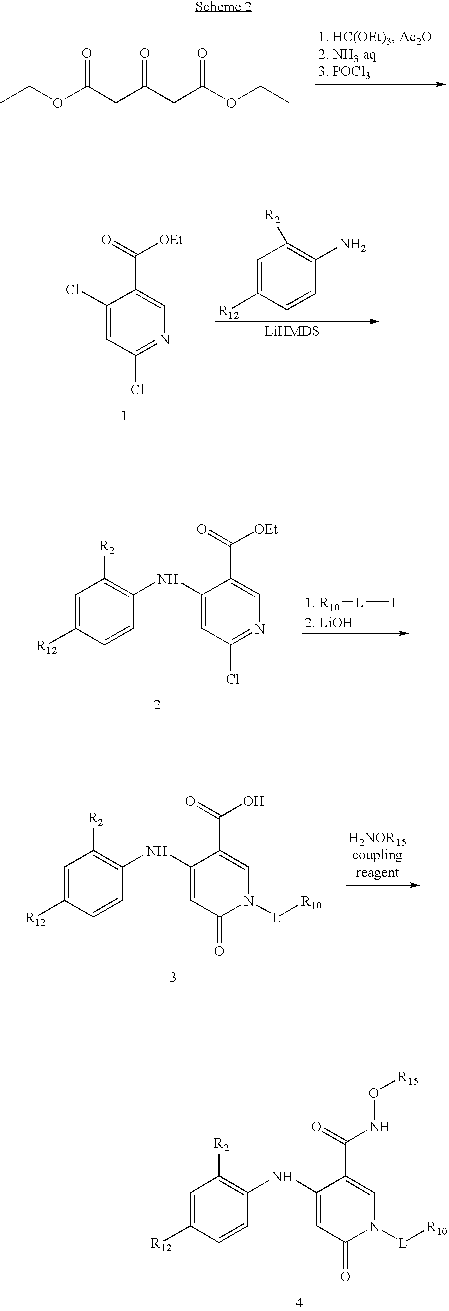

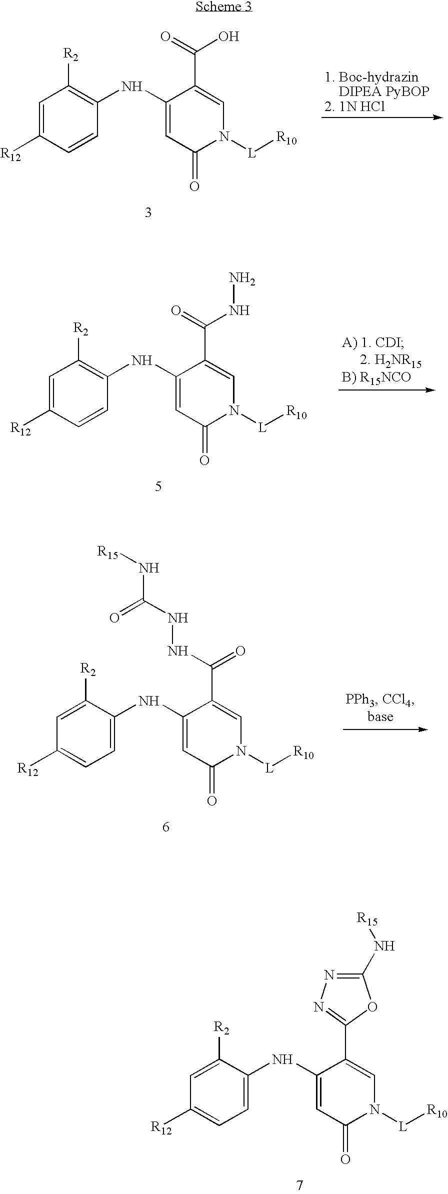

According to a literature procedure diethyl 1,3-acetonedicarboxylate is heated with triethyl orthoformate and acetic anhydride and subsequently treated with ammonia as shown in scheme 2. The resulting product is converted into ethyl 4,6-dichloronicotinate (1) by heating with phosphoryl chloride. This procedure has been previously described in the literature (DenHertog, Recl Trav Chim Pays-Bas 1946, 65, 129-140). Compound 1 is then reacted with an appropriately substituted aniline in an inert solvent, preferable THF, by addition of a base, preferably but not limited to LiHMDS, to yield ethyl 6-chloro-4-arylaminonicotinate 2. Heating with an appropriately substituted alkyl iodide leads to an intermediate pyridinium compound which is directly converted into pyridone carboxylate 3. In the next step compound 3 is coupled with an O-alkyl hydroxalamine using an appropriate coupling reagent including but not limited to PyBOP; PyBroP, EDC or DCC in a suitable organic solvents like for example DMF, THF or DCM to yield hydroxamate 4.

Scheme 3 illustrates the preparation of compounds of the present invention where W is heterocyclic. In step 1, 4-anilino pyridone compound 3 is reacted with BOC-hydrazine, DIPEA and a coupling reagent as PyBOP, for example. The product is then deprotected with hydrochloric acid at elevated temperatures to give acylhydrazide 5. Reaction of 5 with CDI or any suitable carbonate equivalent in a preferred solvent such as DMF or DCM and subsequent reaction with a substitued amine in ethanol gives hydrazine carboxamide 6. Alternatively, hydrazide 5 can be reacted with an appropriately substituted isocyanate to yield compound 6. Cyclization to 7 is achieved by addition of triphenyl phosphine, CCl₄ and a base such as triethylamine or DIPEA in an inert solvent like DCM.

Suitable anilines, alkyliodides, O-alkyl hydroxylamines, and isocyanates are commercially available from Sigma-Aldrich Chemie GmbH, Munich, Germany or from Acros Organics, Belgium or from Fisher Scientific GmbH, 58239 Schwerte, Germany or can be routinely prepared by procedures described in “March's Advanced Organic Chemistry: Reactions, Mechanisms, and Structure”, 5th Edition; John Wiley & Sons. O-[(4S)2,2-Dimethyl-[1,3]dioxolan-4-ylmethyl]-hydroxylamine and O-[(4R)2,2-dimethyl-[1,3]dioxolan-4-ylmethyl]-hydroxylamine are prepared according to a procedure described in WO02/06213 A2.

Compounds with other variants in the position of W can be prepared by derivatizing the COOH group appropriately as known to the person skilled in the art as described in Theophil Eicher, Siegfried Hauptmann “The Chemistry of Heterocycles; Structures, Reactions, Synthesis and Application”, 2^{nd} edition, Wiley-VCH 2003. The introduction of alternative heterocyclic or heteroaryl groups is exemplified e.g. in WO 03/077855 and WO 01/05391.

Unless otherwise noted, all non-aqueous reactions were carried out either under an argon or nitrogen atmosphere with commercial dry solvents. Compounds were purified using flash column chromatography using Merck silica gel 60 (230-400 mesh), or by reverse phase preparative HPLC using a Reprosil-Pur ODS3, 5 μm, 20×125 mm column with Shimadzu LC8A-Pump and SPD-10Avp UV/Vis diode array detector. The ¹H-NMR spectra were recorded on a Varian VXR-S (300 MHz for ¹H-NMR) using d₆-dimethylsulfoxide or d₄-methanol as solvent; chemical shifts are reported in ppm relative to tetramethylsilane. Analytical LC/MS was performed using Reprosil-Pur ODS3, 5 μM, 1×60 mm columns at a flow rate of 250 μl/min, sample loop 2.5 μl; retention times are given in minutes. Methods are: (I) runs on a LC10Advp-Pump (Shimadzu) with SPD-M10Avp UV/Vis diode array detector and QP2010 MS-detector in ESI+ modus with UV-detection at 214, 254 and 275 nm with a gradient of 15-95% acetonitrile (B) in water (A) (0.1% formic acid), 5 min. linear gradient; (II) idem but linear gradient 8 min 1-30% B; (III) idem but linear gradient 8 min 10-60% B; (IV) idem but linear gradient 8min 15-99% B; (V) idem but linear gradient 10 min 5-95% B; (VI) idem but linear gradient 10 min 10-95% B; (VII) idem but linear gradient 5 min 10-90% B.

### EXAMPLES

The examples below are intended to further illustrate particular embodiments of the invention and are not intended to limit the scope of the specification in any way.

### Example 1

### 4-(4-Bromo-2-methyl-phenylamino)-1-methyl-6-oxo-1,6-dihydro-pyridine-3-carboxylic acid (3a)

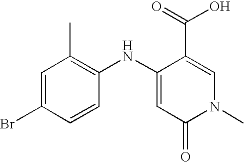

Compound 3a is synthesised in a multistep procedure as outlined in scheme 2.

Step A

Ethyl 4,6-dichloronicotinate (1) is synthesised according to a literature procedure (DenHertog, Recl Trav Chim Pays-Bas 1946, 65, 129-140): Diethyl acetone dicarboxylate (10 g, 49 mmol, purchased from Sigma-Aldrich) is treated with triethylorthoformiate (7 g) and acetic anhydride (10 g) at 130° C. for 1 h and the volatiles are removed in vacuo. The mixture is cooled to 0° C. and conc. Ammonia (14 ml) is added slowly. The precipitate is filtered, washed with 25% hydrochloric acid and recrystallised from hot ethanol to give 5 g (27 mmol, 56% yield) of ethyl 4,6 dihydroxynicotinate. After treating the intermediate with an excess of POCl₃ at 160° C. for 1 h with microwave irradiation, the mixture is hydrolysed on ice and extracted with ethyl acetate to give crude 1 which is purified by flash chromatography using silica and 0-20% ethyl acetate in cyclohaxane as eluent, yielding 3.3 g pure 1 (15 mmol, 56% yield).

Step B

Ethyl 4,6-dichloronicotinate (1) (2.0 g, 9.1 mmol) and 4-bromo-2-methylaniline (1.7 g, 9.1 mmol) are dissolved in dry THF (20 ml) under argon and the mixture is cooled to −78° C. A solution of LiHMDS (1.0M in THF, 32 ml) is slowly added and the reaction mixture is allowed to warm to ambient temperature. After 18 h the reaction is quenched by adding dilute hydrochloric acid (1.0M, 20.0 ml) and the mixture is extracted with DCM (3×60ml). The combined organic extracts are concentrated in vacuo and the crude material is purified by flash chromatography using silica gel and a gradient of 0-10% ethylacetate in cyclohexane as eluent to give pure 4-(4-bromo-2-methyl-phenylamino)-6-chloro-nicotinic acid ethyl ester (2a) (900 mg, 27% yield).

Step C

4-(4-Bromo-2-methyl-phenylamino)-6-chloro-nicotinic acid ethyl ester (2a) (250 mg, 0.68 mmol) and iodomethane (337 μl, 5.4 mmol) are dissolved in dry DCE (8 ml) under argon and the mixture is heated at 140° C. for 100 min with microwave irradiation and the volatiles are removed in vacuo. LiOH (101 mg, 4.2 mmol) is added and the mixture is heated for 10 min at 140° C. with microwave irradiation. The volatiles are evaporated under reduced pressure and the crude material is purified by flash chromatography using silica gel and a gradient of 0-10% MeOH in DCM/formic acid 0.5% as eluent to give 157 mg of pure 4-(4-bromo-2-methyl-phenylamino)-1-methyl-6-oxo-1,6-dihydro-pyridine-3-carboxylic acid (3a)

LC-MS method (V) rt 5.06 min; m/z 337, 339 [M+H]+, Br pattern.

1H-NMR (300 MHz, DMSO-d6) δ=2.2 (s, 3H), 3.35 (s, 3H), 5.45 (s, 1H), 7.20-7.26 (d, 1H), 7.35-7.42 (d, 1H), 7.48 (s, 1H), 8.15 (s, 1H).

### Example 2

### 4-(4-Bromo-2-methyl-phenylamino)-1-methyl-6-oxo-1,6-dihydro-pyridine-3-carboxylic acid hydroxyamide (4a)

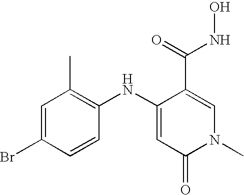

4-(4-Bromo-2-methyl-phenylamino)-1-methyl-6-oxo-1,6-dihydro-pyridine-3-carboxylic acid (3a) (50 mg, 0.15 mmol) is dissolved in 2.5 ml dry DMF and DIPEA (0.59 mmol, 103 μl), PyBOP (0.222 mmol, 116 mg) and hydroxylamine hydrochloride (0.37 mmol, 26 mg) is added. The mixture is stirred for 16 h at 50° C. and for 48 h at ambient temperature. The volatiles are removed in vacuo and the crude material is purified by preparative HPLC to give 14.2 mg of 4a.

LC-MS method (V): rt 5.06 min; m/z 352, 354 [M+H]⁺, Br pattern.

¹H-NMR (300 MHz, MeOH-d₄) δ=2.15 (s, 3H), 3.38 (s, 3H), 5.40 (s, 1H), 7.07-7.12 (d, 1H), 7.28-7.33 (d, 1H), 7.40 (s, 1H), 7.86 (b, 1H).

### Example 3

### 4-[(4-Bromo-2-methylphenyl)amino]-N-{[(2S)-2,3-dihydroxypropyl]oxy}-1-methyl-6-oxo-1,6-dihydropyridine-3-carboxamide (4b)

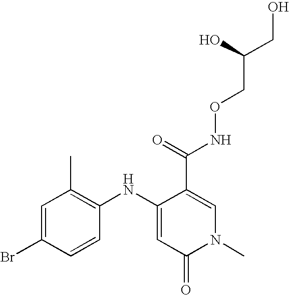

4-(4-Bromo-2-methyl-phenylamino)-1-methyl-6-oxo-1,6-dihydro-pyridine-3-carboxylic acid (3a) (0.30 mmol, 100 mg) is dissolved in dry DMF (5 ml) followed by the addition of DIPEA (1.2 mmol, 153 μl), PyBOP (0.45 mmol, 231 mg) and O-[(4S)2,2-dimethyl-[1,3]dioxolan-4-ylmethyl]-hydroxylamine (0.45 mmol, 65.5 mg). The mixture is stirred for 16 h at 60° C. and the volatiles are removed in vacuo. The crude material is redissolved in MeOH (12 ml) and H₂O (1.4 ml), Dowex50X8 (95 mg) is added and the mixture is heated for 10 min at 120° C. with microwave irradiation. After filtration the volatiles are removed in vacuo and the product is purified by preparative HPLC to give 9.7 mg of pure product 4b.

LC-MS method (VI): rt 2.62 min m/z 426, 428 [M+H]⁺, Br pattern.

¹H-NMR (300 MHz, MeOH-d₄) δ=2.15 (s, 3H), 3.39 (s, 3H), 3.50-3.54 (m, 2H), 3.79-3.90 (m, 2H), 3.96-4.01 (m, 1H), 5.38 (s, 1H), 7.07-7.11 (d, 1H), 7.29-7.33 (dd, 1H), 7.41 (s, 1 H), 7.95 (s, 1 H).

### Example 4

### 5-(5-Allylamino-[1,3,4]oxadiazol-2-yl)-4-(4-bromo-2-methyl-phenylamino)-1-methyl-1H-pyridin-2-one (7a)

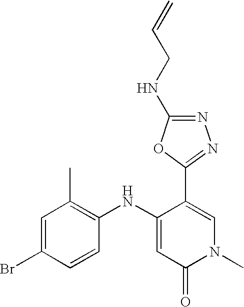

Compound 7a is synthesised in a multistep procedure as outlined in scheme 3.

Step A

4-(4-Bromo-2-methyl-phenylamino)-1-methyl-6-oxo-1,6-dihydro-pyridine-3-carboxylic acid (3a) (245 mg, 0.73 mmol) is dissolved in 7 ml dry DMF followed by the addition of DIPEA (391 μl, 2.2 mmol), PyBOP (0.95 mmol, 492 mg) and N-t-butoxycarbonylhydrazide (1.45 mmol, 192 mg) and the mixture is stirred for 4 h at 60° C. DMF is removed in vacuo and the crude material is dissolved in ethylacetate, washed with saturated bicarbonate solution (3×), H₂O (1×) and brine (1×) and dried (Na₂SO₄). The volatiles are removed in vacuo and the product is purified by flash chromatography using silica gel and a gradient of 0-50% ethylacetate in cyclohexane to give an N-Boc-protected intermediate (213 mg, 65% yield). This Intermediate is dissolved in THF (4 ml) and a solution of HCl in dioxane (1M) is added (3 ml). The reaction mixture is stirred for 3.5 h at ambient temperature, the volatiles are removed in vacuo. The crude product is redissolved in dry THF (2 ml), DIPEA (0.94 mmol, 169 μl) is added followed by allylisocyanate (0.52 mmol, 46 μl). The solution is stirred at ambient temperature for 2 h and the volatiles are removed in vacuo to give crude 6a, which is used without purification in step B.

### N-allyl-2-({4-[(4-bromo-2-methylphenyl)amino]-1-methyl-6-oxo-1,6-dihydropyridin-3-yl}carbonyl)hydrazinecarboxamide (6a):

LC-MS method (VII): rt 2.65 min m/z 351, 353 [M+H]⁺, Br pattern.

Step B

N-allyl-2-({4-[(4-bromo-2-methylphenyl)amino]-1-methyl-6-oxo-1,6-dihydropyridin-3-yl}carbonyl)hydrazinecarboxamide (6a) (0.472 mmol, 205) mg is dissolved in dry DCM (10ml) and CCl₄ (1.89 mmol,183 μl), triethylamine (0.71 mmol, 99 μl) and PPh₃ (0.73 mmol,192 mg) are added and the solution is heated for 20 min at 100° C. with microwave irradiation. The volatiles are removed in vacuo and the product is purified by preparative HPLC to give pure 7a.

LC-MS method (III): rt 7.8 min m/z 416, 418 [M+H]⁺, Br pattern.

¹H-NMR (400 MHz, DMSO-d₆) δ=2.21 (s, 3H), 3.42 (s, 3H), 3.90 (t, J=5.6 Hz, 2H); 5.15 (d, J=11.6 Hz, 1H), 5.27 (d, J=17.2 Hz, 1H), 5.32 (s, 1H), 5.88-5.98 (m, 1H), 7.28 (d, J=8.6 Hz), 7.47 (dd, J=2.0 Hz, J=8.1 Hz, 1H), 7.60 (d, J=2.0 Hz), 8.03 (t, J=6.1 Hz, 1H), 8.15 (s, 1H), 9.03 (s, 1H).

### Example 5

### 4-(4-Iodo-2-methyl-phenylamino)-1-methyl-6-oxo-1,6-dihydro-pyridine-3-carboxylic acid (3b)

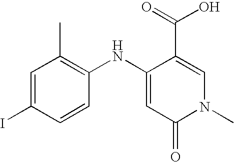

Ethyl 4,6-dichloronicotinate (1) (3.2 g, 14.5 mmol) and 4-iodo-2-methylaniline (3.0 g, 13.6 mmol) are dissolved in dry THF (15 ml) under argon and the mixture is cooled to −78° C. A solution of LiHMDS (1.0M in THF, 48 ml) is slowly added and the reaction mixture is allowed to warm to ambient temperature. After 18 h the reaction is quenched by adding dilute hydrochloric acid (1.0M, 30.0 ml) and the mixture is extracted with DCM (3×80 ml). The combined organic extracts are concentrated in vacuo and the crude material is purified by flash chromatography using silica gel and a gradient of 0-20% ethylacetate in cyclohexane as eluent to give pure 4-(4-iodo-2-methyl-phenylamino)-6-chloro-nicotinic acid ethyl ester (2b) (1.2 g, 20% yield).

Ester 2b (350 mg, 0.84 mmol) and iodomethane (1.5 ml) are dissolved in dry DCE (8 ml) under argon and the mixture is heated at 140° C. for 1.5 h with microwave irradiation and the volatiles are removed in vacuo. LiOH (81 mg, 3.36 mmol) is added and the mixture is heated for 15 min at 140° C. with microwave irradiation. The volatiles are evaporated under reduced pressure 1N HCl is added (10 ml) and extraxted with DCM. The organic phase is dried over Na₂SO₄ and evaporated and the crude material is purified by preparative RP18-HPLC to give pure 4-(4-iodo-2-methyl-phenylamino)-1-methyl-6-oxo-1,6-dihydro-pyridine-3-carboxylic acid (3b) (44 mg, 0.11 mmol, 14% yield). LC-MS method (III) rt 7.07 min; m/z 385 [M+H]⁺.

¹H-NMR (400 MHz, DMSO-d₆) δ=2.16 (s, 3H), 3.38 (s, 3H), 5.24 (s, 1H), 7.11 (d, J=8.1 Hz, 1H), 7.59 (d, J=8.6 Hz, 1H), 7.71 (s, 1H), 8.47 (s, 1H), 9.6 (b, 1H).

### Example 6

### 4-[(4-Iodo-2-methylphenyl)amino]-N-{[(2S)-2,3-dihydroxypropyl]oxy}-1-methyl-6-oxo-1,6-dihydropyridine-3-carboxamide (4c)

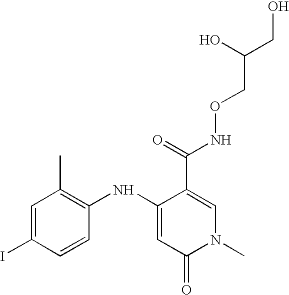

4-(4-Iodo-2-methyl-phenylamino)-1-methyl-6-oxo-1,6-dihydro-pyridine-3-carboxylic acid (3b) (0.467 mmol, 180 mg) is dissolved in dry DMF (2 ml) followed by the addition of DIPEA (0.70 mmol, 121 μl), PyBOP (0.70 mmol, 365 mg) and O-[(4S)2,2-dimethyl-[1,3]dioxolan-4-ylmethyl]-hydroxylamine (0.935 mmol, 129 mg). The mixture is stirred for 16 h at 60° C., aqueous phosphate buffer (pH 7, 15 ml) is added and extracted with DCM. The combined organic phases are washed with brine, dried (Na₂SO₄) end evaporated. The crude material is purified by flash chromatography using silica and a 0-10% gradient of methanol in DCM to yield 85 mg (35% yield) of acetonide-protected 4c. Methanol (2 ml), water (0.2 ml) and Dowex50X8 (20 mg) is added and the mixture is heated for 10 min at 120° C. with microwave irradiation. After filtration the volatiles are removed in vacuo and the product is purified by preparative HPLC to give 26 mg of pure product 4c.

LC-MS method (III): rt 5.45 min, m/z 474 (M+H]⁺.

¹H-NMR (400 MHz, DMSO-d₆) δ=2.16 (s, 3H), 3.17 (d, J=5.5 Hz, 3H), 3.38-3.41 (m, 1H), 3.71-3.81 (m, 2H), 3.95 (dd, J=3.5 Hz, J=9.6 Hz, 1H), 4.10 (dd, J=5.0 Hz, J=10.6 Hz, 1H), 4.63 (b, 1H), 4.88 (b, 1H), 5.31 (s, 1H), 7.09 (d, J=8.1 Hz, 1H); 7:58 (d, J=8.6 Hz, 1H), 7.70 (s, 1H), 8.10 (s, 1H), 9.21 (b′s, 1H).

Assay

The activity of the compounds of the present invitation may be determined by the following procedure: Inhibition of human MEK1 kinase activity was monitored with a homogenous, fluorescence based assay. The assay uses time resolved fluorescence resonance energy transfer to probe for phosphorylation of ERK1 by MEK1. The assay is carried out in low volume 96 well microtiterplates. In a total volume of 15 μl, compounds are incubated with 100 nM MEK1, 15 μM ATP, 300 nM ERK2 employing a buffer containing 20 mM TRIS/HCl, 10 mM MgCl2, 100 μM NaVO4, 1 mM DTT, and 0.005% Tween 20 (pH 7.4). After two hours, 5 nM Europium-anti-PY20 (Perkin Elmer) and 50 nM Anti-GST-Allophycocyanin (CisBio) in buffer containing 50 mM EDTA and 0,05% BSA are added and the reaction incubated for one hour in the dark. Time-resolved fluorescence is measured using a LJL-Analyst (Molecular Devices) with an excitation wavelength of 340 nm and an emission wavelength of 665 nm. The final concentration of DMSO is 2%. To assess the inhibitory potential of the compounds, IC50-values were determined.

In this assay compounds of the invention exhibited IC50s within certain ranges. The following compounds exemplify such activity with “+” meaning 1 μM<IC50≦10 μM and “++” IC50≦1 μM

| Compound # | Activity |
|---|---|
| 3a | ++ |
| 4a | ++ |
| 4b | ++ |
| 7a | ++ |
| 3b | ++ |
| 4c | ++ |

Assay 2: Tumor Cell Proliferation Assays (ATP Lite)

Murine colon C26, human melanoma A375 and human melanoma Mel5 cells were plated in 96 well Corning white plates (1500 cells/well for C26, and 2000 cells/well for A375, and MiaPaCa-2) and cultured overnight at 37° C. in 5% CO2. Inhibitors were serially diluted in 100% DMSO and subsequently added to cells to reach a final concentration of 0.25% DMSO. The cells were incubated for 4 days in the presence of test compounds in cell growth media (DMEM with 10% fetal bovine serum, 2 mM glutamine for C26, and MiaPaCa-2, and RPMI with 10% fetal bovine serum, 2 mM glutamine for A375). Cell proliferation was quantitated using the ATP lite cell proliferation kit (Packard). Inhibition of cell proliferation is shown in Table 2. Columns 2-4 show the concentration of compounds required to induce 50% cell death (IC50 in μM) of human endometriotic cells. With “+” meaning 100 μM<IC50≦10 μM and “++” IC50≦1 μM and “n.d.” means not determined.

Assay 3: Microsomal Stability Assay

Compounds were tested on their stability in human, rat and mouse liver microsomal preparations (HLM, RLM and MLM respectively). At a final concentration of 3 μM, compounds were incubated at 37° C. with 0.5 mg/ml human, rat or mouse liver microsomes in a buffer containing 50 mM phosphate, pH 7.4 and 2 mM NADPH. Pooled human liver microsomes or pooled male rat liver microsomes (Sprague Dawley) were obtained from NatuTec (Frankfurt, Germany). Incubations without NADPH served as negative controls. Reactions were stopped after 0, 15, 30, 45 or 60 min by the addition of acetonitrile and microsomes were pelleted by centrifugation (10 min at 6200× g). Supernatants were analyzed by HPLC regarding the concentration of mother compound. Finally, the half-life of compounds in the regarding microsomal preparation was calculated. Results are shown in Table 2. Wherein “+” means t_{1/2} of 1-30 min, “++” means t_{1/2} of 31-120 min and “+++” means t_{1/2} of >120 min.

TABLE 2
Results of inhibition of tumor cell proliferation and
microsomal stability

| Compound | C26 cells | Mel5 cells | A375 cells | HLM t½ | RLM t½ |
|---|---|---|---|---|---|
| No. | IC50 [uM] | IC50 [uM] | IC50 [uM] | [min] | [min] |
| 3a | + | + | + | n.d. | n.d. |
| 4a | + | + | + | n.d. | n.d. |
| 4b | + | + | + | +++ | +++ |
| 7a | n.d. | n.d. | n.d. | ++ | ++ |
| 4c | + | n.d. | + | +++ | +++ |

1-12. (canceled)

13. A compound of formula (I), 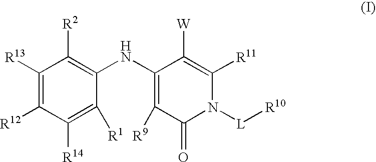
a pharmaceutically acceptable salt, solvate or prodrug thereof,
wherein:
R¹, R², R⁹, R¹¹, R¹², R¹³ and R¹⁴ are independently selected from: hydrogen, halogen, cyano, nitro, azido, —OR³, —C(O)R³,—C(O)OR³, —NR⁴C(O)OR⁶, —OC(O)R³, —NR⁴S(O)ⱼR⁶, —S(O)ⱼNR³R⁴, —S(O)ⱼNR⁴C(O)R³, —C(O)NR⁴S(O)ⱼR⁶, —S(O)ⱼR⁶, —NR⁴C(O)R³, —C(O)NR³R⁴, —NR⁵C(O)NR³R⁴, —NR⁵C(NCN)NR³R⁴, —NR³R⁴, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR⁴R⁵)ₘ-aryl, aryl, arylalkyl, heteroaryl, heteroarylalkyl, heterocyclyl, heterocyclylalkyl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl, —NR⁴(CR⁴R⁵)ₘ-heterocyclyl, and —S(C₁-C₂ alkyl) substituted with 1 to 5 fluorines, wherein each alkyl, alkenyl, alkynyl, cycloalkyl, aryl, heteroaryl and heterocyclyl is optionally and independently substituted;
R¹⁰ is selected from: hydrogen, —OR³, —C(O)R³,—C(O)OR³, —NR⁴C(O)OR⁶, —OC(O)R³, —NR⁴S(O)ⱼR⁶, —S(O)ⱼNR³R⁴, —S(O)ⱼNR⁴C(O)R³, —C(O)NR⁴S(O)ⱼR⁶, S(O)ⱼR⁶, —NR⁴C(O)R³, —C(O)NR³R⁴, —NR⁵C(O)NR³R⁴, —NR⁵C(NCN)NR³R⁴, —NR³R⁴; —S(O)ⱼ(C₁-C₆ alkyl), —S(O)ⱼ(CR⁴R⁵)ₘ-aryl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵ )ₘ-heteroaryl, —O(CR⁴R⁵)ₘ-heterocyclyl, —NR⁴(CR⁴R⁵)ₘ-heterocyclyl, and —S(C₁-C₂ alkyl) substituted with 1 to 5 flourines, wherein each aryl, heteroaryl and heterocyclyl is optionally and independently substituted;
L is selected from: C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, heterocyclylalkyl, where each alkyl, alkenyl, alkynyl, cycloalkyl, aryl, heteroaryl and heterocyclyl, wherein each group is optionally and independently substituted;
or L and R¹⁰ are together hydrogen;
R³ is selected from: hydrogen, trifluoromethyl, C₁-C₁₀ alkyl, C₂₋₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl, each of which is optionally and independently substituted;
R⁴ is selected from: hydrogen and C₁-C₆ alkyl, wherein the alkyl may be optionally substituted; or
R³ and R⁴ can be taken together with the atom to which they are attached to form a 4 to 10 membered heteroaryl or heterocyclic ring, each of which is optionally and independently substituted;
R⁵ is selected from: hydrogen and C₁-C₆ alkyl, wherein the alkyl may be optionally substituted; or
R⁴ and R⁵ can be taken together with the atom to which they are attached to form a 4 to 10 membered carbocyclic, heteroaryl or heterocyclic ring, each of which is optionally and independently substituted;
R⁶ is selected from: trifluoromethyl,C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is optionally and independently substituted;
W is selected from: 1) heteroaryl containing 1-4 heteroatoms or heterocyclyl containing 1-4 heteroatoms, each of which is optionally substituted by 1 to 5 substituents ZR₁₅; and 2) —C(O)OR¹⁵, —C(O)NR⁴R¹⁵, —C(O)NR⁴OR¹⁵, —C(O)(C₃-C₁₀ cycloalkyl), —C(O)(C₂-C₁₀ alkyl), —C(O)(aryl), —C(O)(heteroaryl), —C(O)(heterocyclyl), S(O)ⱼNR⁴R¹⁵, S(O)ⱼNR⁴OR¹⁵, —S(O)ⱼNR⁴C(O)R¹⁵, or —C(O)NR⁴S(O)ⱼR⁶, whereby R⁴ and R¹⁵ are as defined herein, or taken together may form a 3 to 7 membered ring with 1 or 2 nitrogen atoms and optionally an oxygen atom;
Z is a bond, NR¹⁶, O, NR¹⁶SO₂ or S;
R¹⁵ is independently selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, wherein each alkyl, alkenyl, alkynyl, cycloalkyl, aryl, heteroaryl and heterocyclyl is optionally and indepdently substituted;
R¹⁶ is selected from: hydrogen and C₁-C₁₀ alkyl;
or R¹⁵ and R¹⁶ taken together form a 4 to 10 membered cyclic ring with 1 or 2 nitrogen atoms and optionally an oxygen atom, said ring being optionally substituted;
m is an integer from 0 to 5; and
j is 1 or 2.

14. The compound according to claim 13 wherein:
R¹, R², R⁹, R¹¹ are independently selected from: hydrogen, halogen, C₁-C₄ alkyl, C₃-C₄ cycloalkyl, C₂-C₄ alkenyl, C₂-C₄ alkynyl, cyano, nitro, OR³ and NR³R⁴, wherein each alkyl, alkenyl, alkynyl, cycloalkyl is optionally and independently substituted with one to five halogens;
R¹⁰ is selected from: hydrogen, —OR³, —NR⁴C(O)R³, —C(O)NR³R⁴, and —NR³R⁴;
L is C₁-C₅ alkyl;
or L and R¹⁰ are together hydrogen;
R¹² is selected from: hydrogen, halo, C₁-C₁₀ alkyl, C₃-C₁₀ cycloalkyl, C₂-C₁₀ alkenyl, C₂-C₁₀ alkynyl, cyano, nitro, azido, NR⁴SO₂R⁶, SO₂NR³R⁴, SO₂R⁶, C(O)NR³R⁴, —S(O)ⱼNR⁴C(O)R³, —C(O)NR⁴S(O)ⱼR⁶, OR³, NR³R⁴ and —S(C₁-C₂ alkyl) substituted with 1 to 5 fluorines, wherein each alkyl, alkenyl, alkynyl, cycloalkyl, aryl, heteroaryl and heterocyclyl is optionally and independently substituted;
R¹³ and R¹⁴ are independently selected from: H, F, Cl, C₁-C₄ alkyl, C₃-C₄ cycloalkyl, C₂-C₄ alkenyl, and C₂-C₄ alkynyl, wherein each alkyl, alkenyl, cycloalkyl, alkynyl is optionally and independently substituted with one to five halogens;
W is selected from: 1) heteroaryl containing 1-4 heteroatoms or heterocyclyl containing 1-4 heteroatoms, each of which is optionally substituted by 1 to 3 substituents ZR¹⁵; or 2) —C(O)OR¹⁵, —C(O)NR⁴R¹⁵, —C(O)NR⁴OR¹⁵, —C(O)(C₃-C₁₀ cycloalkyl), —C(O)(C₂-C₁₀ alkyl), —S(O)ⱼNR⁴C(O)R¹⁵, —C(O)NR⁴S(O)ⱼR⁶, —S(O)ⱼNR⁴R¹⁵ or S(O)ⱼNR⁴OR¹⁵;
Z is selected from: NR¹⁶, NR¹⁶SO₂ and O;
R¹⁵ is selected from: hydrogen, C₁-C₄ alkyl, C₁-C₄ alkenyl, and C₄-C₆ cycloalkylalkyl, wherein each alkyl or alkenyl is optionally and independently substituted by 1 or 2 -OH, —O—C₁-C₄ alkyl or —NR′R″ groups;
R¹⁶ is selected from: hydrogen and C₁-C₄ alkyl; and
R′ and R″ are each independently selected from hydrogen, C₁-C₄ alkyl, C₂-C₄ alkenyl, aryl and arylalkyl.

15. The compound according to claim 13 wherein:
R¹ is selected from: H and F;
R² is selected from: F, Cl, Me, wherein the methyl group is optionally substituted with one to three fluorines;
R⁹ is selected from: H, F, and Cl;
R¹⁰ is selected from: hydrogen, —OR³, and —NR³R⁴;
L is selected from: ethylene, n-propylene and n-butylene;
or L and R¹⁰ together are methyl;
R¹¹ is selected from: H, F, Cl, Br, Me, and OMe, wherein the methyl groups are optionally substituted with one to three fluorines;
R¹² is selected from: H, F, Cl, Br, I, nitro, methyl, ethyl, n-propyl, i-propyl, cyclopropyl, —SCF₃, —SCHF₂, —SCH₂F, —SO₂NR₃R₄, —C(O)NR³R⁴ or —OMe, wherein the methyl groups are optionally substituted with one to three fluorines;
wherein R³ and R⁴ are each independently: 1) C₁-C₆ alkyl, optionally substituted by 1 or 2 alkyl amino; or O-alkyl; or
R³ and R⁴ form together a cyclic ring with 1 or 2 nitrogen atoms and optionally an oxygen atom, said ring being optionally substituted by 1 or 2 alkyl amino or O-alkyl groups;
R¹³ and R¹⁴ are independently selected from H and F;
W is selected from —C(O)NR⁴OR¹⁵ or SO₂NR⁴OR¹⁵; or W is: 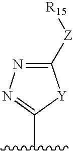
wherein
Z is NR¹⁶;
R¹⁵ is C₁-C₄ alkyl or C₁-C₄ alkenyl optionally substituted with 1 to 3 substituents selected from: OH, O-Me, NH₂, N(methyl)₂ and N(ethyl)₂;
R¹⁶ is hydrogen or C₁-C₄ alkyl; or R¹⁶ and R¹⁵ taken together form a 4 to 10 membered cyclic ring with 1 or 2 nitrogen atoms and optionally an oxygen atom, said ring being optionally substituted by 1 or 2 alkyl amino, amino, hydroxy or O-alkyl groups; and
Y is O, S or NR′.

16. The compound according to claim 13 wherein:
W is selected from —C(O)NR⁴OR¹⁵ and SO₂NR⁴OR¹⁵, or W is: 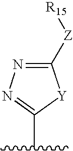
wherein
R⁴ is hydrogen;
Z is NH;
R¹⁵ is selected from: C₁-C₄ alkyl and C₁-C₄ alkenyl, each of which may be further substituted by 1 or 2 groups selected from: OH, O—C₁-C₄ alkyl and NR′R″;
R′ and R″ are independently hydrogen, methyl or ethyl; and
Y is O.

17. The compound according to claim 13 wherein L and R¹⁰ together are methyl.

18. A method for treating a mammal with a hyperproliferative disease or a disorder mediated by aberrant proliferation comprising administering to the mammal a therapeutically effective amount of a compound according to Formula I: 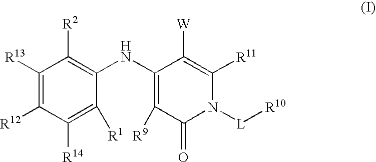
a pharmaceutically acceptable salt, solvate or prodrug thereof,
wherein:
R¹, R², R⁹, R¹¹, R¹², R¹³ and R¹⁴ are independently selected from: hydrogen, halogen, cyano, nitro, azido, —OR³, —C(O)R³, —C(O)OR³, —NR⁴C(O)OR⁶, —OC(O)R³, —NR⁴S(O)ⱼR⁶, —S(O)ⱼNR³R⁴, —S(O)ⱼNR⁴C(O)R³, —C(O)NR⁴S(O)ⱼR⁶, —S(O)ⱼR⁶, —NR⁴C(O)R³, —C(O)NR³R⁴, —NR⁵C(O)NR³R⁴, —NR⁵C(NCN)NR³R⁴, —NR³R⁴, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR⁴R⁵)ₘ-aryl, aryl, arylalkyl, heteroaryl, heteroarylalkyl, heterocyclyl, heterocyclylalkyl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl, —NR⁴(CR⁴R⁵)ₘ-heterocyclyl, and —S(C₁-C₂ alkyl) substituted with 1 to 5 fluorines, wherein each alkyl, alkenyl, alkynyl, cycloalkyl, aryl, heteroaryl and heterocyclyl is optionally and independently substituted;
R¹⁰ is selected from: hydrogen, —OR³, —C(O)R³, —C(O)OR³, —NR⁴C(O)OR⁶, —OC(O)R³, —NR⁴S(O)ⱼR⁶, —S(O)ⱼNR³R⁴, —S(O)ⱼNR⁴C(O)R³, —C(O)NR⁴S(O)ⱼR⁶, S(O)ⱼR⁶, —NR⁴C(O)R³, —C(O)NR³R⁴, —NR⁵C(O)NR³R⁴, —NR⁵C(NCN)NR³R⁴, —NR³R⁴; —S(O)ⱼ(C₁-C₆ alkyl), —S(O)ⱼ(CR⁴R⁵)ₘ-aryl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl, —NR⁴(CR⁴R⁵)ₘ-heterocyclyl, and —S(C₁-C₂ alkyl) substituted with 1 to 5 flourines, wherein each aryl, heteroaryl and heterocyclyl is optionally and independently substituted;
L is selected from: C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, heterocyclylalkyl, where each alkyl, alkenyl, alkynyl, cycloalkyl, aryl, heteroaryl and heterocyclyl, wherein each group is optionally and independently substituted;
or L and R¹⁰ are together hydrogen;
R³is selected from: hydrogen, trifluoromethyl, C₁-C₁₀ alkyl, C₂-₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl, each of which is optionally and independently substituted;
R⁴is selected from: hydrogen and C₁-C₆ alkyl, wherein the alkyl may be optionally substituted; or
R³ and R⁴ can be taken together with the atom to which they are attached to form a 4 to 10 membered heteroaryl or heterocyclic ring, each of which is optionally and independently substituted;
R⁵ is selected from: hydrogen and C₁-C₆ alkyl, wherein the alkyl may be optionally substituted; or
R⁴ and R⁵ can be taken together with the atom to which they are attached to form a 4 to 10 membered carbocyclic, heteroaryl or heterocyclic ring, each of which is optionally and independently substituted;
R⁶ is selected from: trifluoromethyl,C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is optionally and independently substituted;
W is selected from: 1) heteroaryl containing 1-4 heteroatoms or heterocyclyl containing 1-4 heteroatoms, each of which is optionally substituted by 1 to 5 substituents ZR₁₅; and 2) —C(O)OR¹⁵, —C(O)NR⁴R¹⁵, —C(O)NR⁴OR¹⁵, —C(O)(C₃-C₁₀ cycloalkyl), —C(O)(C₂-C₁₀ alkyl), —C(O)(aryl), —C(O)(heteroaryl), —C(O)(heterocyclyl), S(O)ⱼNR⁴R¹⁵, S(O)ⱼNR⁴OR¹⁵, —S(O)ⱼNR⁴C(O)R¹⁵, or —C(O)NR⁴S(O)ⱼR⁶, whereby R⁴ and R¹⁵ are as defined herein, or taken together may form a 3 to 7 membered ring with 1 or 2 nitrogen atoms and optionally an oxygen atom;
Z is a bond, NR²⁶, O, NR¹⁶SO₂ or S;
R¹⁵ is independently selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, wherein each alkyl, alkenyl, alkynyl, cycloalkyl, aryl, heteroaryl and heterocyclyl is optionally and indepdently substituted;
R¹⁶ is selected from: hydrogen and C₁-C₁₀ alkyl;
or R¹⁵ and R¹⁶ taken together form a 4 to 10 membered cyclic ring with 1 or 2 nitrogen atoms and optionally an oxygen atom, said ring being optionally substituted;
m is an integer from 0 to 5; and
j is 1 or 2.

19. A method of treating a mammal with a hyperproliferative disease related to the hyperactivity of MEK or diseases modulated by the MEK cascade in mammals, comprising administering to the mammal a therapeutically effective amount of a compound according to Formula I: 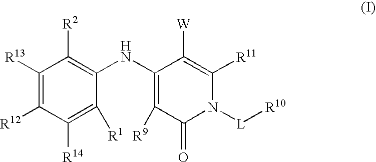
a pharmaceutically acceptable salt, solvate or prodrug thereof,
wherein:
R¹, R², R⁹, R¹¹, R¹², R¹³ and R¹⁴ are independently selected from: hydrogen, halogen, cyano, nitro, azido, —OR³, —C(O)R³, —C(O)OR³, —NR⁴C(O)OR⁶, —OC(O)R³, —NR⁴S(O)ⱼR⁶, —S(O)ⱼNR³R⁴, —S(O)ⱼNR⁴C(O)R³, —C(O)NR⁴S(O)ⱼR⁶, —S(O)ⱼR⁶, —NR⁴C(O)R³, —C(O)NR³R⁴, —NR⁵C(O)NR³R⁴, —NR⁵C(NCN)NR³R⁴, —NR³R⁴, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR⁴R⁵)ₘ-aryl, aryl, arylalkyl, heteroaryl, heteroarylalkyl, heterocyclyl, heterocyclylalkyl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl, —NR⁴(CR⁴R⁵)ₘ-heterocyclyl, and —S(C₁-C₂ alkyl) substituted with 1 to 5 fluorines, wherein each alkyl, alkenyl, alkynyl, cycloalkyl, aryl, heteroaryl and heterocyclyl is optionally and independently substituted;
R¹⁰ is selected from: hydrogen, —OR³, —C(O)R³, —C(O)OR³, —NR⁴C(O)OR⁶, —OC(O)R³, —NR⁴S(O)ⱼR⁶, —S(O)ⱼNR³R⁴, —S(O)ⱼNR⁴C(O)R³, —C(O)NR⁴S(O)ⱼR⁶, S(O)ⱼR⁶, —NR⁴C(O)R³, —C(O)NR³R⁴, —NR⁵C(O)NR³R⁴, —NR⁵C(NCN)NR³R⁴, —NR³R⁴;
—S(O)ⱼ(C₁-C₆ alkyl), —S(O)ⱼ(CR⁴R⁵)ₘ-aryl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl, —NR⁴(CR⁴R⁵)ₘ-heterocyclyl, and —S(C₁-C₂ alkyl) substituted with 1 to 5 flourines, wherein each aryl, heteroaryl and heterocyclyl is optionally and independently substituted;
L is selected from: C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, heterocyclylalkyl, where each alkyl, alkenyl, alkynyl, cycloalkyl, aryl, heteroaryl and heterocyclyl, wherein each group is optionally and independently substituted;
or L and R¹⁰ are together hydrogen;
R³ is selected from: hydrogen, trifluoromethyl, C₁-C₁₀ alkyl, C₂-₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl, each of which is optionally and independently substituted;
R⁴ is selected from: hydrogen and C₁-C₆ alkyl, wherein the alkyl may be optionally substituted; or
R³ and R⁴ can be taken together with the atom to which they are attached to form a 4 to 10 membered heteroaryl or heterocyclic ring, each of which is optionally and independently substituted;
R⁵ is selected from: hydrogen and C₁-C₆ alkyl, wherein the alkyl may be optionally substituted; or
R⁴ and R⁵ can be taken together with the atom to which they are attached to form a 4 to 10 membered carbocyclic, heteroaryl or heterocyclic ring, each of which is optionally and independently substituted;
R⁶ is selected from: trifluoromethyl,C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is optionally and independently substituted;
W is selected from: 1) heteroaryl containing 1-4 heteroatoms or heterocyclyl containing 1-4 heteroatoms, each of which is optionally substituted by 1 to 5 substituents ZR₁₅; and 2) —C(O)OR¹⁵, —C(O)NR⁴R¹⁵, —C(O)NR⁴OR¹⁵, —C(O)(C₃-C₁₀ cycloalkyl), —C(O)(C₂-C₁₀ alkyl), —C(O)(aryl), —C(O)(heteroaryl), —C(O)(heterocyclyl), S(O)ⱼNR⁴R¹⁵, S(O)ⱼNR⁴OR¹⁵, —S(O)ⱼNR⁴C(O)R¹⁵, or —C(O)NR⁴S(O)ⱼR⁶, whereby R⁴ and R¹⁵ are as defined herein, or taken together may form a 3 to 7 membered ring with 1 or 2 nitrogen atoms and optionally an oxygen atom;
Z is a bond, NR¹⁶, O, NR¹⁶SO₂ or S;
R¹⁵ is independently selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, wherein each alkyl, alkenyl, alkynyl, cycloalkyl, aryl, heteroaryl and heterocyclyl is optionally and indepdently substituted;
R¹⁶ is selected from: hydrogen and C₁-C₁₀ alkyl;
or R¹⁵ and R¹⁶ taken together form a 4 to 10 membered cyclic ring with 1 or 2 nitrogen atoms and optionally an oxygen atom, said ring being optionally substituted;
m is an integer from 0 to 5; and
j is 1 or 2.

20. The method of claim 19 wherein the disease is selected from: cancer, inflammation, pancreatitis or kidney disease, pain, benign hyperplasia of the skin, restenosis, prostate, diseases related to vasculogenesis or angiogenesis, tumor angiogenesis, psoriasis, eczema, sclerodema, diabetes, diabetic retinopathy, retinopathy of prematurity, age-related macular degeneration, hemangioma, glioma, melanoma and Kaposi's sarcoma.

21. The method of claim 20 wherein the disease is cancer or inflammation.

22. The method of claim 21 wherein the type of cancer is selected from: ovarian, breast, lung, pancreatic, prostate, colon and epidermoid.

23. The method of claim 21 wherein the type of inflammation is selected from: rheumatoid arthritis, inflammatory bowel disease, and atherosclerosis.

24. A pharmaceutical composition comprising a compound according to claim 1 and a pharmaceutically acceptable carrier.
